(19) United States
(12) Patent Application Publication
(10) Pub. No.: US 2024/0175080 A1
(43) Pub. Date: May 30, 2024

(54) SPATIAL ANALYSIS TO DETECT SEQUENCE VARIANTS

(71) Applicant: 10x Genomics, Inc., Pleasanton, CA (US)

(72) Inventors: Christina Galonska, Stockholm (SE); Marco Mignardi, Sundbyberg (SE)

(21) Appl. No.: 18/404,317

(22) Filed: Jan. 4, 2024

Related U.S. Application Data
(63) Continuation of application No. 18/179,795, filed on Mar. 7, 2023, now Pat. No. 11,959,130.
Application No. 18/179,795 is a continuation of application No. 17/551,980, filed on Dec. 15, 2021, now Pat. No. 11,608,520.
Application No. 17/551,980 is a continuation of application No. PCT/US21/33649, filed on May 21, 2021.
(60) Provisional application No. 63/029,110, filed on May 22, 2020.

(51) Int. Cl. C12Q 1/6837; C12Q 1/6841; C12Q 1/6855

(52) U.S. Cl. CPC C12Q 1/6837; C12Q 1/6841; C12Q 1/6855

(57) ABSTRACT

Provided herein are methods of detecting an analyte of interest to interrogate spatial gene expression in a sample.

### CROSS-REFERENCE TO RELATED APPLICATIONS

This application is a continuation of U.S. patent application Ser. No. 18/179,795, filed on Mar. 7, 2023, which is a continuation of U.S. patent application Ser. No. 17/551,980, now U.S. Pat. No. 11,608,520, filed Dec. 15, 2021, which is a continuation of International Application PCT/US2021/033649, with an international filing date of May 21, 2021, which claims priority to U.S. Provisional Patent Application No. 63/029,110, filed May 22, 2020, which is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogencity has been previously studied using techniques that provide data for a small handful of analytes in the context of an intact tissue or a portion of a tissue, or provide a lot of analyte data for single cells, but fail to provide information regarding the position of the single cell in a parent biological sample (e.g., tissue sample).

Generally, spatial analysis takes advantage of targeting a particular analyte in a sample using a capture probe that has a poly(A) mRNA-like tail. However, this approach is capable of detecting a high number of off-target analytes. One option to overcome this issue is to utilize multiple oligonucleotides that target adjacent or nearby complementary sequences though a process called RNA-templated ligation (RTL), or simply templated ligation. However, even methods involving templated ligation can include detection of off-target analytes. Thus, there remains a need to develop an alternative to poly(A) mRNA-like capture of target analytes that is capable of detecting an analyte(s) in an entire transcriptome.

### SUMMARY

This disclosure features improved methods of RNA-template ligation (RTL), or templated ligation to increase detection of a target analyte. Templated ligation seeks to increase target-specific detection of an analyte through hybridization of multiple (e.g., two) oligonucleotides, or probes, that are ligated together to one oligonucleotide product that can be detected by a capture probe on a spatial array. This disclosure features improvements to templated ligation that increase efficiency of target detection. In some instances, this disclosure provides methods that can detect targets that vary by as small as a single nucleotide (e.g., in the setting of a single nucleotide polymorphism (SNP)). In some instances, the ligation step of templated ligation only occurs when the SNP (or point mutation) is present. This is because, in one instance, the disclosure provides an invader assay that occurs in the presence of the SNP (or point mutation). In this way, non-specific off-targets ligation products are not created, thereby increasing templated ligation efficiency and specificity. In some instances, this disclosure allows for increased efficiency of templated ligation using oligonucleotides that are complementary to sequences on an analyte, wherein the sequences are not exactly adjacent to one another on the analyte (i.e., there is a sequence between the two complementary sequences on the analyte). In some instances, templated ligation efficiency is increased through use of a ligase that is pre-activated with ATP before the ligation step. Finally, in some instances, templated ligation efficiency is increased using a third oligonucleotide probe that is complementary to the intervening sequence between the two templated ligation oligonucleotides. Thus, the disclosure provides multiple embodiments that increase templated ligation specificity and efficiency such that the detection of a particular analyte of interest can be more easily detected compared to settings in which the methods of the disclosure are not utilized.

Thus, disclosed herein are methods for determining the presence or absence of a genetic variant in an analyte at a spatial location in a biological sample comprising: (a) contacting the biological sample with a first probe oligonucleotide and a second probe oligonucleotide, wherein the first probe oligonucleotide and the second probe oligonucleotide each comprise a sequence that is substantially complementary to adjacent sequences of the analyte, wherein the first probe oligonucleotide and the second probe oligonucleotide are capable of forming an invasive cleavage structure in the presence of the analyte, wherein the second probe oligonucleotide comprises a capture probe binding domain; (b) hybridizing the first probe oligonucleotide and the second probe oligonucleotide to the analyte; (c) ligating the first probe oligonucleotide and the second probe oligonucleotide, thereby creating a ligated probe; (d) releasing the ligated probe from the analyte; (e) contacting the ligated probe with a substrate comprising a plurality of capture probes, wherein a capture probe of the plurality comprises a spatial barcode and the capture domain; (f) hybridizing the ligated probe to the capture domain; and (g) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the analyte in the biological sample.

In some instances, the methods further include cleaving a portion of the second probe oligonucleotide. In some instances, the first probe oligonucleotide comprises at least one nucleotide that is complementary to a wild-type sequence relative to the genetic variant. In some instances, the methods further include second probe oligonucleotide comprises a sequence of non-complementary nucleotides. In some instances, the methods further include second probe oligonucleotide comprises at least one nucleotide complementary to the at least one genetic variant of the analyte.

Also disclosed herein are methods for determining the presence or absence of a genetic variant in an analyte at a spatial location in a biological sample comprising: (a) contacting the biological sample with a first probe oligonucleotide and a second probe oligonucleotide, wherein the first probe oligonucleotide and the second probe oligonucleotide each comprise a sequence that is substantially complementary to adjacent sequences of the analyte; wherein the first probe oligonucleotide and the second probe oligonucleotide are capable of forming an invasive cleavage structure in the presence of the genetic variant; and wherein: (i) the first probe oligonucleotide further comprises at least one nucleotide that is complementary to a wild-type sequence of the genetic variant; and (ii) the second probe oligonucleotide further comprises: a sequence of non-complementary nucleotides; a nucleotide complementary to the genetic variant; and a capture probe binding domain; (b) hybridizing the first probe oligonucleotide and the second probe oligonucleotide to the analyte; (c) cleaving the sequence of non-complementary nucleotides when the genetic variant is present; (d) ligating the first probe oligonucleotide and the second probe oligonucleotide, thereby creating a ligated probe; (e) releasing the ligated probe from the analyte; (f) contacting the ligated probe with a substrate comprising a plurality of capture probes, wherein a capture probe of the plurality comprises a spatial barcode and the capture domain; (g) hybridizing the ligated probe to the capture domain; and (h) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the analyte in the biological sample.

In some instances, the first probe oligonucleotide comprises a sequence that is substantially complementary to a sequence 3′ of the genetic variant.

In some instances, the first probe oligonucleotide comprises a sequence that is substantially complementary to a sequence 5′ of the genetic variant. In some instances, the second probe oligonucleotide comprises a sequence substantially complementary to a sequence 5′ to the genetic variant. In some instances, the second probe oligonucleotide comprises a sequence substantially complementary to a sequence 3′ to the genetic variant.

In some instances, the second probe oligonucleotide comprises from 5′ to 3′: a sequence of non-complementary nucleotides, one or more nucleotides complementary to the genetic variant, a sequence substantially complementary to a sequence 5′ of the genetic variant, and a capture probe binding domain that is capable of binding to a capture domain of a capture probe.

In some instances, the analyte comprises one or more single nucleotide variants.

In some instances, the cleaving step comprises providing an endonuclease. In some instances, the endonuclease cleaves the invasive cleavage structure. In some instances, the endonuclease cleaves a portion of the first probe oligonucleotide. In some instances, the endonuclease cleaves the at least one nucleotide that is complementary to a wild-type sequence of the analyte of the first probe oligonucleotide. In some instances, the endonuclease cleaves a portion of second probe oligonucleotide. In some instances, the endonuclease cleaves the sequence of non-complementary nucleotides of the second probe. In some instances, the endonuclease is Flap endonuclease 1 (FEN1). In some instances, the endonuclease is selected from a Thermus aquaticus (Taq) DNA polymerase, a Thermus thermophilus (Tth) polymerase, Xeroderma pigmentosum group G (XPG) protein, Rad2 from budding yeast, AtRAD2 from Arabidopsis, or Rad13 in fission yeast, or derivatives thereof.

In some instances, the first probe oligonucleotide further comprises a first moiety. In some instances, the first moiety is a first fluorophore. In some instances, the first fluorophore emits a first signal when the first probe oligonucleotide is cleaved. In some instances, the second probe oligonucleotide further comprises a second moiety. In some instances, the second moiety is a second fluorophore. In some instances, the second fluorophore emits a second signal when the second probe oligonucleotide is cleaved. In some instances, the first signal and the second signal emit at different wavelengths.

In some instances, the ligating comprises ligating the cleaved first probe oligonucleotide and the cleaved second probe oligonucleotide using enzymatic or chemical ligation. In some instances, the method comprises repeating steps (a)-(c) above with a second set of first probe oligonucleotides and second set of second probe oligonucleotides. In some instances, the at least one genetic variant is a single nucleotide polymorphism (SNP). In some instances, the at least one genetic variant is a nucleotide point mutation. In some instances, the at least one genetic variant comprises at least two, at least three, at least four, at least five, or more genetic variants.

Also disclosed herein are methods for identifying a location of an analyte in a biological sample, the methods comprising: (a) contacting the biological sample with a first probe oligonucleotide and a second probe oligonucleotide, wherein the first probe oligonucleotide comprises a sequence substantially complementary to a sequence 3′ of a target sequence, the second probe oligonucleotide comprises: a sequence of non-complementary nucleotides; a sequence substantially complementary to a sequence 5′ of the target sequence, and a capture probe binding domain, and the target sequence is at least one nucleotide; (b) hybridizing the first probe oligonucleotide and the second probe oligonucleotide to the analyte; (c) extending the first probe oligonucleotide to generate an extended first probe oligonucleotide; (d) cleaving the sequence of non-complementary nucleotides; (e) ligating the extended first probe oligonucleotide and the second probe oligonucleotide, thereby creating a ligated probe that is substantially complementary to the analyte; (f) releasing the ligated probe from the analyte; (g) contacting the ligated probe with a substrate, wherein the capture probe is affixed to the substrate, wherein the capture probe comprises a spatial barcode and the capture domain; (h) allowing the capture probe binding domain of the ligated probe to specifically bind to the capture domain; and (i) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the location of the analyte in the biological sample.

In some instances, the target sequence comprises one nucleotides, two nucleotides, three nucleotides, four nucleotides, five nucleotides, at least 10 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some instances, the second probe oligonucleotide comprises a sequence of non-complementary nucleotides. In some instances, the second probe oligonucleotide comprises from 5′ to 3′: a sequence of non-complementary nucleotides, a sequence substantially complementary to a sequence 5′ of the target sequence, and a capture probe binding domain that is capable of binding to a capture domain of a capture probe. In some instances, the extending the first probe oligonucleotide comprises a nucleic acid extension reaction. In some instances, the extending the first probe oligonucleotide comprises reverse transcription. In some instances, the extending the first probe oligonucleotide comprises using a reverse transcriptase. In some instances, the extending the first probe oligonucleotide comprises using a Moloney Murine Leukemia Virus (M-MulV) reverse transcriptase. In some instances, the extending the first probe oligonucleotide generates a sequence that is complementary to the analyte. In some instances, the extending the first probe oligonucleotide generates a sequence that is adjacent to the second probe oligonucleotide.

In some instances, the ligation step comprises ligating the extended first probe oligonucleotide and the second probe oligonucleotide using enzymatic or chemical ligation.

In some instances, the cleaving comprises an endonuclease. In some instances, the endonuclease is Flap endonuclease 1 (FEN1). In some instances, the endonuclease is selected from a Thermus aquaticus (Taq) DNA polymerase, a Thermus thermophilus (Tth) polymerase, Xeroderma pigmentosum group G (XPG) protein, Rad2 from budding yeast, AtRAD2 from Arabidopsis, or Rad13 in fission yeast, or derivatives thereof. In some instances, the endonuclease cleaves at least the non-complementary nucleotides from the second probe oligonucleotide.

In some instances, the second probe oligonucleotide further comprises a second moiety. In some instances, the second moiety is a second fluorophore. In some instances, the second fluorophore emits a second signal when the second probe oligonucleotide is cleaved.

In some instances, the ligation step comprises ligating the extended first probe oligonucleotide and the cleaved second probe oligonucleotide using enzymatic or chemical ligation.

Also disclosed herein are methods for identifying a location of an analyte having one or more genetic variants in a biological sample, the methods comprising: (a) contacting the biological sample with a first probe oligonucleotide and a second probe oligonucleotide, wherein the first probe oligonucleotide comprises a sequence substantially complementary to a sequence 3′ of a target sequence, wherein the second probe oligonucleotide comprises a sequence substantially complementary to a sequence 5′ of the target sequence and a capture probe binding domain that is capable of binding to a capture domain of a capture probe, and wherein the complementary sequences to which the first probe oligonucleotide and the second probe oligonucleotide bind are at least one nucleotide away from each other; (b) hybridizing the first probe oligonucleotide and the second probe oligonucleotide to the analyte; (c) contacting the biological sample with a plurality of gap probe oligonucleotides, wherein a gap probe oligonucleotide of the plurality gap probe oligonucleotides comprises a sequence substantially complementary to the target sequence; (d) ligating (i) the first probe oligonucleotide and the 5′ end of a gap probe oligonucleotide and (ii) the second probe oligonucleotide and the 3′ end of the gap probe oligonucleotide, thereby creating a ligated probe that is substantially complementary to the analyte; (e) releasing the ligated probe from the analyte; (f) contacting the ligated probe with a substrate comprising a plurality of capture probes, wherein a capture probe of the plurality comprises a spatial barcode and the capture domain; (g) hybridizing the ligated probe to the capture domain; and (h) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the analyte in the biological sample. In some instances, the method comprises performing step (c) prior to step (a).

In some instances, the target sequence comprises one nucleotides, two nucleotides, three nucleotides, four nucleotides, five nucleotides, at least 10 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, or more nucleotides. In some instances, the target sequence comprises one or more single nucleotide variants compared to a reference target sequence.

In some instances, the plurality of gap probe oligonucleotides comprises a first gap probe oligonucleotide comprising a sequence complementary to a target sequence comprising one or more single nucleotide variants. In some instances, the plurality of gap probe oligonucleotides comprises a second gap probe oligonucleotide comprising a sequence complementary to a reference target sequence.

In some instances, the ligation step comprises ligating the first probe oligonucleotide and the 3′ end of a gap probe oligonucleotide using enzymatic or chemical ligation. In some instances, the ligation step comprises ligating the second probe oligonucleotide and the 5′ end of a gap probe oligonucleotide using enzymatic or chemical ligation. In some instances, the enzymatic ligation utilizes a ligase. In some instances, the ligase is one or more of a T4 RNA ligase (Rn12), a SplintR ligase, a single stranded DNA ligase, or a T4 DNA ligase. In some instances, the ligase is a T4 RNA ligase 2 (Rnl2) ligase. In some instances, the ligase is a pre-activated T4 DNA ligase.

In some instances, the biological sample is a tissue. In some instances, the biological sample is a tissue section. In some instances, the biological sample is a formalin-fixed, paraffin-embedded (FFPE) sample, a frozen sample, or a fresh sample. In some instances, the biological sample is an FFPE sample. In some instances, the analyte is RNA. In some instances, the RNA is an mRNA.

In some instances, the first probe oligonucleotide is at least 70%, at least 75%, at least 80%, at least 85%, at least 90%, at least 95%, at least 96%, at least 97%, at least 98%, at least 99%, or 100% complementary to a sequence of the analyte. In some instances, the second probe oligonucleotide is at least 70%, at least 75%, at least 80%, at least 85%, at least 90%, at least 95%, at least 96%, at least 97%, at least 98%, at least 99%, or 100% complementary to a sequence of the analyte. In some instances, the first probe oligonucleotide comprises at least two ribonucleic acid bases at the 3′ end. In some instances, the first probe oligonucleotide further comprises a functional sequence. In some instances, the functional sequence is a primer sequence. In some instances, the second probe oligonucleotide comprises a phosphorylated nucleotide at the 5′ end.

In some instances, the method further comprises providing a capture probe binding domain blocking moiety that interacts with the capture probe binding domain. In some instances, the method further comprises releasing the capture probe binding domain blocking moiety from the capture probe binding domain prior to contacting the biological sample with the substrate.

In some instances, the capture probe binding domain comprises a homopolymeric sequence. In some instances, the capture probe binding domain comprises a poly(A) sequence or complement thereof. In some instances, the capture probe binding domain blocking moiety comprises a poly-uridine sequence, a poly-thymidine sequence, or both.

In some instances, releasing the poly-uridine sequence from the poly(A) sequence comprises denaturing the ligated probe or contacting the ligated probe with an endonuclease or exonuclease. In some instances, the releasing step comprises removing the ligated probe from the analyte. In some instances, the releasing step comprises contacting the ligated probe with an endoribonuclease. In some instances, the endoribonuclease is one or more of RNase H, RNase A, RNase C, or RNase I. In some instances, the endoribonuclease is RNAseH. In some instances, the RNase H comprises RNase H1, RNase H2, or RNase H1 and RNase H2.

In some instances, the method further comprises contacting the biological sample with a permeabilization reagent. In some instances, the biological sample is contacted with the permeabilization reagent before releasing the ligated probe from the analyte. In some instances, the permeabilization reagent is an endopeptidase.

In some instances, the method further comprises amplifying the ligated probe prior to contacting the biological sample with a substrate.

In some instances, the capture probes are on a support. In some instances, the support is a slide.

In some instances, the determining step comprises amplifying all or part of the ligated probe specifically bound to the capture domain. In some instances, the amplifying is isothermal. In some instances, the amplifying is not isothermal. In some instances, an amplifying product comprises (i) all or part of sequence of the ligated probe specifically bound to the capture domain, or a complement thereof, and (ii) all or a part of the sequence of the spatial barcode, or a complement thereof. In some instances, the determining step comprises sequencing.

Also disclosed herein are kits. In some instances, the disclosure provides a kit comprising: (a) an array comprising a plurality of capture probes; (b) a plurality of probes comprising a first probe oligonucleotide and a second probe oligonucleotide, wherein the first probe oligonucleotide and the second probe oligonucleotide each comprise a sequence that is substantially complementary to adjacent sequences of the analyte, wherein the first probe oligonucleotide and the second probe oligonucleotide are capable of forming an invasive cleavage structure in the presence of the analyte, wherein the second probe oligonucleotide comprises a capture probe binding domain; and (c) instructions for performing any of the methods disclosed herein.

In some instances, the disclosure provides a kit comprising: (a) an array comprising a plurality of capture probes; (b) a plurality of probes comprising a first probe oligonucleotide and a second probe oligonucleotide, wherein the first probe oligonucleotide and the second probe oligonucleotide each comprise a sequence that is substantially complementary to sequences of the analyte where there is a gap between the sequences of the analyte, wherein the first probe oligonucleotide and the second probe oligonucleotide are capable of forming an invasive cleavage structure in the presence of the analyte, wherein the second probe oligonucleotide comprises a capture probe binding domain; and (c) instructions for performing any of the methods disclosed herein.

In some instances, the first probe oligonucleotide in the kits further comprises at least one nucleotide that is complementary to a wild-type sequence of a genetic variant. In some instances, the second probe oligonucleotide in the kits further comprises: (i) a sequence of non-complementary nucleotides; (ii) a nucleotide complementary to a genetic variant; and (iii) a capture probe binding domain. In some instances, the kit further comprises an endonuclease. In some instances, the endonuclease is Flap endonuclease 1 (FEN1). In some instances, the kits further comprise a reverse transcriptase. In some instances, the reverse transcriptase comprises a Moloney Murine Leukemia Virus (M-MulV). In some instances, the kits further comprise a ligase. In some instances, the ligase is T4 RNA ligase (Rnl2) or T4 DNA ligase. In some instances, the second probe comprises a preadenylated phosphate group at its 5′ end and a first probe comprises at least two ribonucleic acid bases at the 3′ end.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce spatially-barcoded cells or cellular contents.

FIG. 7A shows a schematic of an example workflow for genetic variant detection using templated ligation.

FIG. 7B shows a schematic of an example workflow for genetic variant detection using templated ligation using gap filing.

FIG. 7C shows a schematic of an example workflow for genetic variant detection using templated ligation with exemplary gap probes. Wt mRNA: wild type mRNA.

FIG. 8 shows a polyacrylamide gel (PAGE gel) displaying results of 5′ Flap cleavage mediated by: (1) Tth polymerase, (2) Taq polymerase or (3) FEN1. RHS: right hand probe.

FIGS. 9A-9B show schematics of an example workflow for genetic variant detection using templated ligation.

FIG. 9C shows a PAGE gel displaying results of 5′ Flap cleavage by FEN1 endonuclease for the workflows in FIG. 9A (left panel) and FIG. 9B (right panel)

FIG. 10 shows a schematic of an example workflow for templated ligation using preactivated T4 DNA ligase.

### DETAILED DESCRIPTION

### I. Introduction

Disclosed herein are methods and compositions predicated on using templated ligation to detect targets that vary by as small as a single nucleotide (e.g., in the setting of a single nucleotide polymorphism (SNP)). In some instances, the ligation step occurs when the SNP (or point mutation) is present as provided by this disclosure. In this way, non-specific off-targets ligation products are greatly minimized or absent, thereby increasing templated ligation efficiency and specificity. Also disclosed herein are methods and compositions that allow for increased efficiency of templated ligation using oligonucleotides that are complementary to sequences on an analyte, wherein the sequences are not exactly adjacent to one another on the analyte (i.e., there is a sequence between the two complementary sequences on the analyte). Also provided herein are methods and compositions designed to increase templated ligation efficiency through use of a ligase that is pre-activated with ATP before the ligation step. Finally, in some instances, templated ligation efficiency is increased using a third oligonucleotide probe that is complementary to the intervening sequence between the two templated ligation oligonucleotides. Thus, the disclosure provides multiple embodiments that increase templated ligation specificity and efficiency such that the detection of a particular analyte of interest can be more easily detected compared to settings in which the methods of the disclosure are not utilized.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3):442-458, 2015; Trejo et al., PLOS ONE 14(2):c0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or cosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

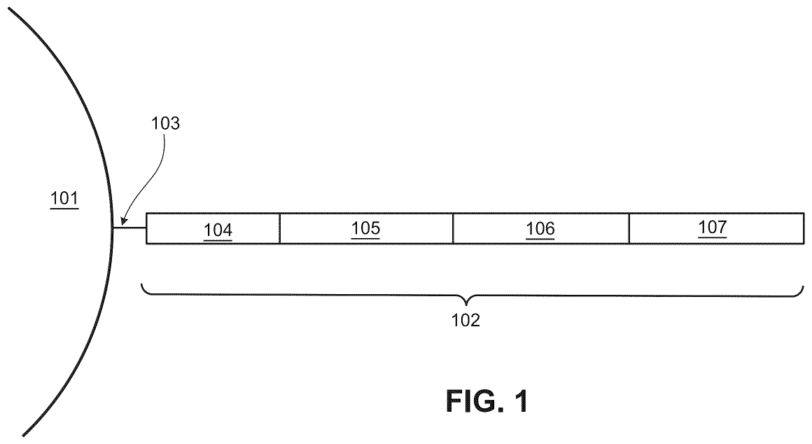

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a RI primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

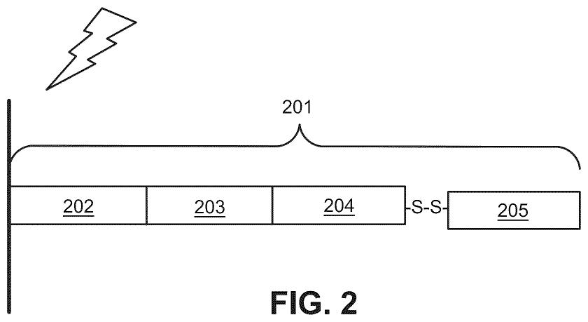

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

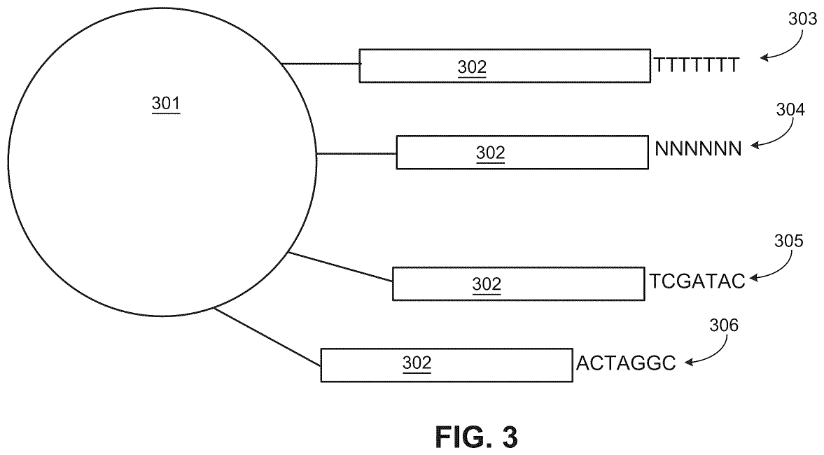

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. Scc, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

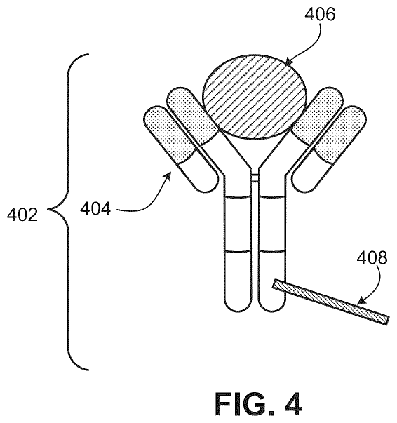

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

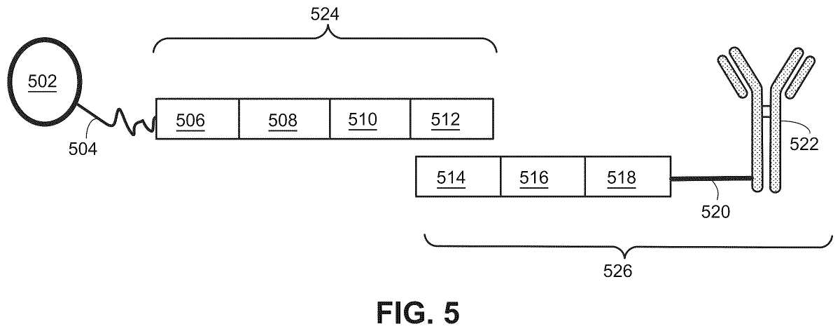

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

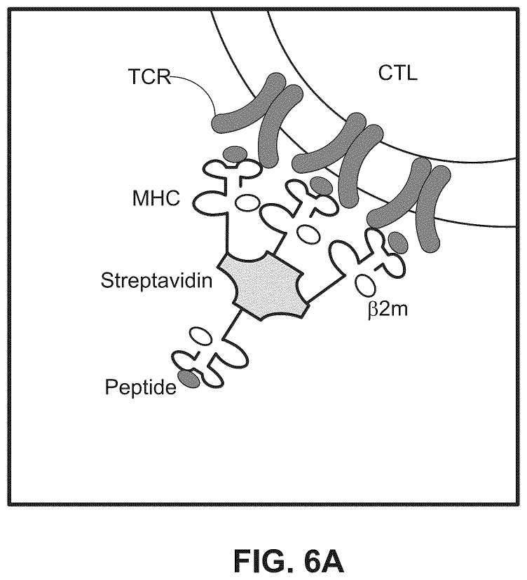

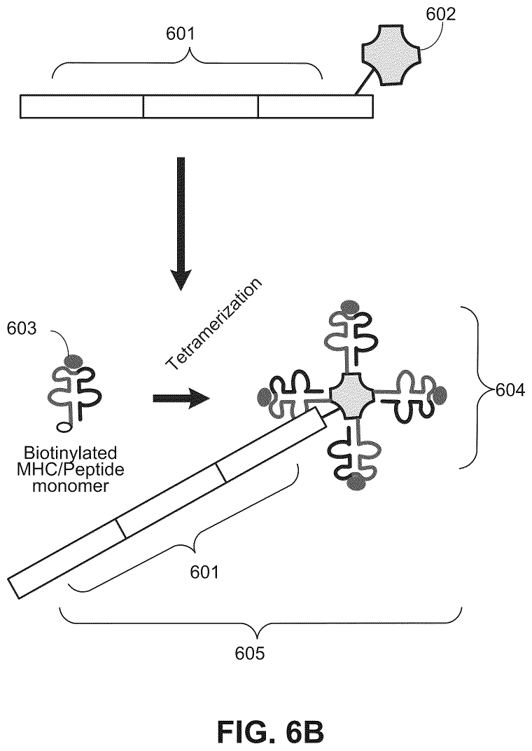

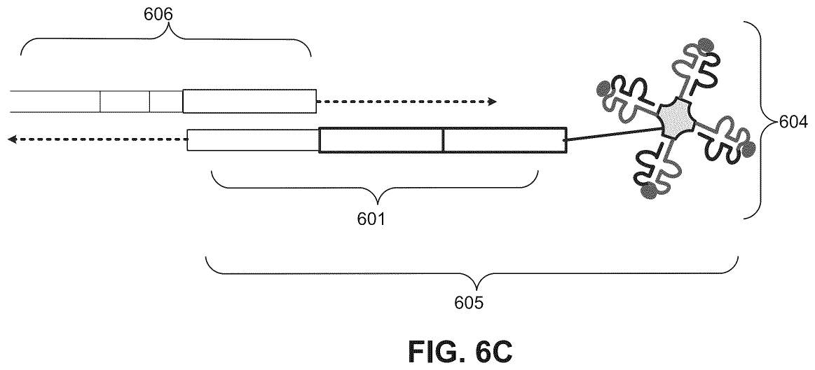

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MHC/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of the corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (scc, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using templated ligation, or RNA-templated ligation (RTL). Methods of templated ligation have been described previously. See, e.g., Credle et al., Nucleic Acids Res. 2017 Aug. 21; 45(14):e128. Typically, templated ligation includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(c)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

II. Templated ligation to Detect Sequence Variants

### (a) Templated Ligation Process and Principles

Templated ligation or RNA-templated ligation (RTL) is a process that includes multiple oligonucleotides (also called “oligonucleotide probes” or simply “probes,” and a pair of probes can be called interchangeably “first probes” and “second probes,” or “first probe oligonucleotides” and “second probe oligonucleotides,”) that hybridize to adjacent complementary analyte (e.g., mRNA) sequences. Upon hybridization, the two oligonucleotides are ligated to one another, creating a ligation product in the event that both oligonucleotides hybridize to their respective complementary sequences. In some instances, at least one of the oligonucleotides includes a sequence (e.g., a poly-adenylation sequence) that can be hybridized to a probe on an array described herein (e.g., the probe comprises a poly-thymine sequence in some instances). In some instances, prior to hybridization of the poly-thymine to the poly(A) sequence, an endonuclease digests the analyte that is hybridized to the ligation product. This step frees the newly formed ligation product to hybridize to a capture probe on a spatial array. In this way, templated ligation provides a method to perform targeted RNA capture on a spatial array.

As an improvement to templated ligation, the present disclosure features the ability to detect modifications or variants to nucleic acid sequences. The present disclosure achieves this ability by using modified probes (i.e., probes with sequences that are complementary to the genetic variants) by designing probes that hybridize to sequences that surround genetic variants.

Targeted RNA capture allows examination of a subset of RNA analytes from the entire transcriptome. One challenge of targeted RNA capture is the ability to accurately detect genetic variants within a subset of RNA analytes. Current challenges are driven by probe design requirements (e.g., a first probe oligonucleotide and/or a second probe oligonucleotides need to incorporate the genetic variation into the probe sequence in order to detect the variation on the complement strand (e.g., the RNA analyte)). To address these challenges, provided herein are methods for determining the presence of a genetic variant or non-variant sequence at a spatial location in a biological sample based (i) on cleavage of overlapping first and second probe oligonucleotide sequences, (ii) gap filling, and (iii) gap probe oligonucleotides. The methods provided herein allow examination of the presence or absence of a genetic variation within a subset of RNA analytes, thereby increasing the resolution of the template ligation technology. In particular, disclosed herein are methods that utilize templated ligation technology to increase the efficiency of targeted RNA capture.

Targeted RNA capture allows for examination of a subset of RNA analytes from the entire transcriptome. In some embodiments, the subset of analytes includes an individual target RNA. In some instances, the presence of the ligation product that is created as a result of the templated ligation methods described herein indicates that the individual target RNA is present. In some instances, the absence of the ligation product that is created as a result of the templated ligation methods described herein indicates that the individual target RNA is not present. In some instances, an absence of the ligation product is because one of the oligonucleotide probes did not hybridize to the analyte. In some instances, an absence of the ligation product is because both (e.g., two) of the oligonucleotide probes did not hybridize to the analyte.

In some embodiments, the subset of analytes detected using methods disclosed herein includes two or more (e.g., 2, 3, 4, 5, 6, 7, 8, 9, 10, or more) targeted RNAs. In some embodiments, the subset of analytes includes one or more mRNAs transcribed by a single gene. In some embodiments, the subset of analytes includes one or more mRNAs transcribed by more than one targeted genes. In some embodiments, the subset of analytes includes one or more mRNA splice variants of one or more targeted genes. In some embodiments, the subset of analytes includes non-polyadenylated RNAs in a biological sample. In some embodiments, the subset of analytes includes detection of mRNAs having one or more (e.g., 2, 3, 4, 5, or more) single nucleotide polymorphisms (SNPs) in a biological sample.

In some embodiments, the subset of analytes includes mRNAs that mediate expression of a set of genes of interest. For example, in some instances, the subset of analytes detected using the templated ligation methods disclosed herein include analytes that are translated into transcription factors that control one or more cellular pathways. In some embodiments, the subset of analytes includes mRNAs that share identical or substantially similar sequences, which mRNAs are translated into polypeptides having similar functional groups or protein domains. In some embodiments, the subset of analytes includes mRNAs that do not share identical or substantially similar sequences, which mRNAs are translated into proteins that do not share similar functional groups or protein domains. In some embodiments, the subset of analytes includes mRNAs that are translated into proteins that function in the same or similar biological pathways. In some embodiments, the biological pathways are associated with a pathologic disease. For example, targeted RNA capture can detect genes that are overexpressed or underexpressed in a cancer sample.

In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, about 1000, or more analytes.

In some embodiments, the subset of analytes detected by targeted RNA capture methods provided herein includes a large proportion of the transcriptome of one or more cells. For example, the subset of analytes detected by targeted RNA capture methods provided herein can include at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the mRNAs present in the transcriptome of one or more cells.

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some embodiments, a biological sample (e.g., tissue section) can be fixed with methanol, stained with hematoxylin and cosin, and imaged. In some embodiments, fixing, staining, and imaging occurs before one or more oligonucleotide probes are hybridized to the sample. Some embodiments of any of the workflows described herein can further include a destaining step (e.g., a hematoxylin and cosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the methods of targeted RNA capture as disclosed herein include hybridization of multiple probe oligonucleotides. In some embodiments, the methods include 2, 3, 4, or more probe oligonucleotides that hybridize to one or more analytes of interest. In some embodiments, the methods include two probe oligonucleotides. In some embodiments, the probe oligonucleotide includes sequences complementary that are complementary or substantially complementary to an analyte. For example, in some embodiments, the probe oligonucleotide includes a sequence that is complementary or substantially complementary to an analyte (e.g., an mRNA of interest (e.g., to a portion of the sequence of an mRNA of interest)). Methods provided herein may be applied to a single nucleic acid molecule or a plurality of nucleic acid molecules. A method of analyzing a sample comprising a nucleic acid molecule may comprise providing a plurality of nucleic acid molecules (e.g., RNA molecules), where each nucleic acid molecule comprises a first target region (e.g., a sequence that is 3′ of a target sequence or a sequence that is 5′ of a target sequence) and a second target region (e.g., a sequence that is 5′ of a target sequence or a sequence that is 3′ of a target sequence), a plurality of first probe oligonucleotides, and a plurality of second probe oligonucleotides.

In some embodiments, the templated ligation methods that allow for targeted RNA capture as provided herein include a first probe oligonucleotide and a second probe oligonucleotide. The first and second probe oligonucleotides each include sequences that are substantially complementary to the sequence of an analyte of interest. By substantially complementary, it is meant that the first and/or second probe oligonucleotide is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an analyte. In some instances, the first probe oligonucleotide and the second probe oligonucleotide hybridize to adjacent sequences on an analyte.

In some embodiments, the first and/or second probe as disclosed herein includes one of at least two ribonucleic acid bases at the 3′ end; a functional sequence; a phosphorylated nucleotide at the 5′ end; and/or a capture probe binding domain. In some embodiments, the functional sequence is a primer sequence. The capture probe binding domain is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or both. In some embodiments, the capture probe binding domain includes a random sequence (e.g., a random hexamer or octamer). In some embodiments, the capture probe binding domain is complementary to a capture domain in a capture probe that detects a particular target(s) of interest.

In some embodiments, a capture probe binding domain blocking moiety that interacts with the capture probe binding domain is provided. In some instances, the capture probe binding domain blocking moiety includes a nucleic acid sequence. In some instances, the capture probe binding domain blocking moiety is a DNA oligonucleotide. In some instances, the capture probe binding domain blocking moiety is an RNA oligonucleotide. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a ligated probe) to a capture probe. In some embodiments, a capture probe binding domain blocking moiety comprises a poly-uridine sequence, a poly-thymidine sequence, or both.

In some embodiments, the first probe oligonucleotide hybridizes to an analyte. In some embodiments, the second probe oligonucleotide hybridizes to an analyte. In some embodiments, both the first probe oligonucleotide and the second probe oligonucleotide hybridize to an analyte. Hybridization can occur at a target having a sequence that is 100% complementary to the probe oligonucleotide(s). In some embodiments, hybridization can occur at a target having a sequence that is at least (e.g., at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe oligonucleotide(s).

After hybridization of the first and second probe oligonucleotides, in some embodiments, the first probe oligonucleotide is extended. After hybridization, in some embodiments, the second probe oligonucleotide is extended. Extending probes can be accomplished using any method disclosed herein. In some instances, a polymerase (e.g., a DNA polymerase) extends the first and/or second oligonucleotide.

In some embodiments, methods disclosed herein include a wash step. In some instances, the wash step occurs after hybridizing the first and the second probe oligonucleotides. The wash step removes any unbound oligonucleotides and can be performed using any technique or solution disclosed herein or known in the art. In some embodiments, multiple wash steps are performed to remove unbound oligonucleotides.

In some embodiments, after hybridization of probe oligonucleotides (e.g., first and the second probe oligonucleotides) to the analyte, the probe oligonucleotides (e.g., the first probe oligonucleotide and the second probe oligonucleotide) are ligated together, creating a single ligated probe that is complementary to the analyte. Ligation can be performed enzymatically or chemically, as described herein.

### (b) Ligation of Hybridized Probe Oligonucleotides

In some instances, the first and second probe oligonucleotides are hybridized to the first and second target regions of the analyte, and the probe oligonucleotides are subjected to a nucleic acid reaction to ligate them together. In some instances, the ligation is enzymatic. In some instances, the ligation reaction catalyzed by a ligase disclosed herein occurs in the following steps. In some instances, the ligase enzyme is pre-activated by charging with ATP. Addition of ATP to ligase enzyme causes formation of an intermediate AMP-enzyme species concomitant with hydrolysis of ATP to yield AMP. In some instances, the pre-activating step does not occur. In some instances, the next step includes methods where the charged AMP-enzyme intermediate binds to the dsDNA (or dsRNA, or RNA/DNA complex) and transfers the AMP moiety to the free 5′ terminal phosphate, to form a high energy 5′-5′ phosphate bond. In some instances, the third step includes methods wherein the enzyme provides the appropriate environment in which the 3′ hydroxyl group of the second strand of DNA (or RNA) is able to attach the high energy 5′-5′ phosphate bond, thereby forming a covalent phosphodiester bond as a product and releasing ligase enzyme and AMP. Free enzyme does not bind the intermediate high energy 5′-5′ phosphate bond species to an appreciable amount. Thus, if the ligase prematurely releases from the duplex after formation of the high energy 5′-5′ phosphate bond, the reaction will typically end and the intermediate will not proceed to the final ligated product.

In some instances, the probes may be subjected to an enzymatic ligation reaction, using a ligase (e.g., T4 RNA ligase (Rn12), a splintR ligase, a single stranded DNA ligase, or a T4 DNA ligase). See, e.g., Zhang L., et al.; Archacal RNA ligase from thermoccocus kodakarensis for template dependent ligation RNA Biol. 2017; 14(1): 36-44, which is incorporated by reference in its entirety, for a description of KOD ligase. In some instances, the ligase is T4 RNA ligase (e.g., Rn12). T4 DNA ligase is an enzyme belonging to the DNA ligase family of enzymes that catalyzes the formation of a covalent phosphodiester bond from a free 3′ hydroxyl group on one DNA molecule and a free 5′ phosphate group of a second, separate DNA molecule, thus covalently linking the two DNA strands together to form a single DNA strand. In some instances, the ligase is splintR ligase. SplintR Ligase, also known as PBCV-1 DNA Ligase or Chorella virus DNA Ligase, efficiently catalyzes the ligation of adjacent, single-stranded DNA oligonucleotides splinted by a complementary RNA strand. In some instances, the ligase is a single-stranded DNA ligase (e.g., CircLigase).

In some embodiments, adenosine triphosphate (ATP) is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers this AMP to the phosphorylated 5′-end at the nick, forming a 5′-5′ pyrophosphate bond. Finally, the ligase catalyzes an attack on this pyrophosphate bond by the OH group at the 3′-end of the nick, thereby sealing it, whereafter ligase and AMP are released. If the ligase detaches from the substrate before the 3′ attack, e.g. because of premature AMP reloading of the enzyme, then the 5′ AMP is left at the 5′-end, blocking further ligation attempts. In some instances, ATP is added at a concentration of about 1 μM, about 10 μM, about 100 μM, about 1000 μM, or about 10000 μM during the ligation reaction.

In some instances, cofactors that aid in joining of the probe oligonucleotides are added during the ligation process. In some instances, the cofactors include magnesium ions (Mg2+). In some instances, the cofactors include manganese ions (Mn2+). In some instances, Mg2+ is added in the form of MgCl2. In some instances, Mn2+ is added in the form of MnCl2. In some instances, the concentration of MgCl2 is at about 1 mM, at about 10 mM, at about 100 mM, or at about 1000 mM. In some instances, the concentration of MnCl2 is at about 1 mM, at about 10 mM, at about 100 mM, or at about 1000 mM.

In some instances, the ligation reaction occurs at a pH in the range of about 6.5 to 9.0, of about 6.5 to 8.0, of about 7.5 to 8.0, of about 7.5, or of about 8.0.

In some embodiments, the ligase is a pre-activated T4 DNA ligase. In templated ligation, T4 DNA ligase will prematurely release from the nicked DNA before the final step, leaving the 5′ to 5′ pyrophosphate bond intermediate species. Released T4 DNA ligase then re-charges itself using available ATP, preventing the enzyme-AMP intermediate species from completing the final step of the reaction, ultimately yielding an accumulation of 5′ to 5′ pyrophosphate bond intermediate product. It has been shown that incubation of the substrate with T4 DNA ligase at a very low ATP concentration improves the ligation efficiency of T4 DNA ligase, but the reaction proceeds very slowly and requires relatively large amounts of enzyme. (See, Nilsson et al., Nucleic Acids Res., 29(2):578 (2001). Moreover, the longer (2 hour or more) incubation period combined with the necessary determination of an optimum ATP concentration can be an obstacle for a number of applications. Thus, disclosed herein are methods of pre-activating T4 DNA ligase with an effective amount of ATP such that ligation efficiency is improved.

In some instances, to generate pre-activated T4 DNA ligase, an effective amount of ATP is added to a solution of DNA ligase. After addition of ATP, the charge of DNA ligase is changed, forming a DNA ligase-AMP intermediate. In some instances, ATP is then depleted from the solution of DNA ligase-AMP intermediate. In some instances, ATP is depleted because the concentration of ATP is low such that DNA ligase utilizes all or most available ATP. In some instances, the solution of DNA ligase-AMP intermediate is added to the hybridized probe oligonucleotides. Methods of utilizing a pre-activated T4 DNA ligase are further disclosed in U.S. Publication No. 20100184618-A1, which is incorporated by reference in its entirety.

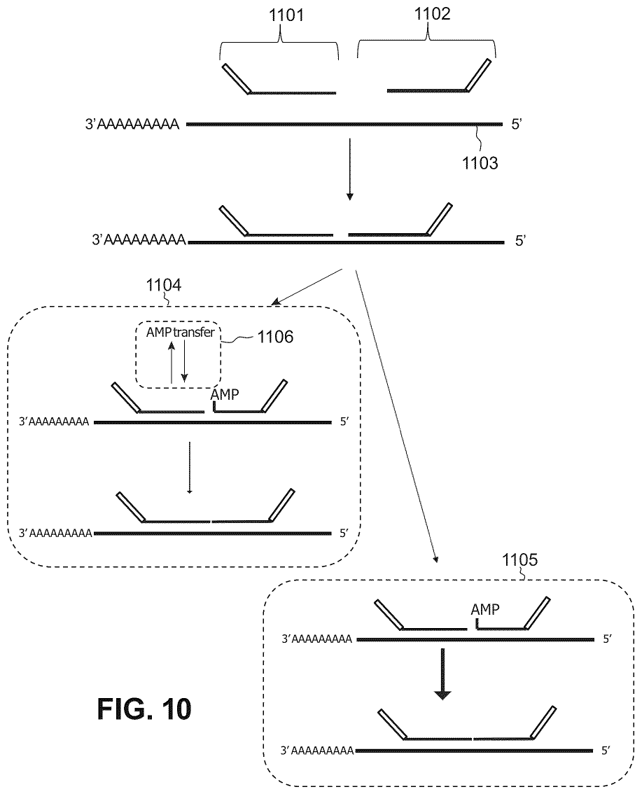

Referring to FIG. 10, a first probe oligonucleotide 1001 and a second probe oligonucleotide 1002 hybridize to an analyte 1003 (e.g., mRNA). In one embodiment 1004, T4 DNA ligase is not pre-incubated with ATP. In such cases, the ATP can be provided along with the T4 DNA ligase. In another embodiment 1005, T4 DNA ligase is pre-activated by incubating the ligase with ATP, as described above. In the setting where no pre-activation occurs, there is an equilibrium 1006 reached where ligation is less efficient compared to a pre-activated T4 DNA ligase (see 1004). In some instances, a pre-activated T4 DNA ligase more efficiency ligates the first and second probe oligonucleotides (see 1005). In some instances, the pre-activated ligase is T4 RNA ligase (Rnl2). In some instances, the pre-activated ligase is splintR ligase. In some instances, the pre-activated ligase is a single-stranded DNA ligase.

Following the enzymatic ligation reaction, the first and second probe oligonucleotides may be considered ligated (e.g., thereby generating a ligated probe or ligated product).

In some embodiments, the probe oligonucleotides (e.g., the first probe oligonucleotide and the second probe oligonucleotide) may each comprise a reactive moiety such that, upon hybridization to the target and exposure to appropriate ligation conditions, the probe oligonucleotides may ligate to one another. In some embodiments, a probe oligonucleotide that includes a reactive moiety is ligated chemically. For example, a probe oligonucleotide capable of hybridizing to a sequence 3′ of a target sequence (e.g., a first target region) of a nucleic acid molecule may comprise a first reactive moiety, and a probe oligonucleotide capable of hybridizing to a sequence 5′ of a target sequence (e.g., a second target region) of the nucleic acid molecule may comprise a second reactive moiety. When the first and second probe oligonucleotides are hybridized to the first and second target regions of the nucleic acid molecule, the first and second reactive moieties may be adjacent to one another. A reactive moiety of a probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phorphorothioate), acids, amines, and phosphates. For example, the first reactive moiety of a first probe oligonucleotide may comprise an azide moiety, and a second reactive moiety of a second probe oligonucleotide may comprise an alkyne moiety. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a thiol-ene reaction; a nucleophilic substation reaction; or another reaction. In some cases, reaction between the first and second reactive moicties may yield a triazole moiety or an isoxazoline moiety. A reaction between the first and second reactive moicties may involve subjecting the reactive moieties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first probe oligonucleotide hybridized to a first target region of the nucleic acid molecule and a second reactive moiety of a third probe oligonucleotide hybridized to a second target region of the nucleic acid molecule may link the first probe oligonucleotide and the second probe oligonucleotide to provide a ligated probe. Upon linking, the first and second probe oligonucleotides may be considered ligated. Accordingly, reaction of the first and second reactive moieties may comprise a chemical ligation reaction such as a copper-catalyzed 5′ azide to 3′ alkyne “click” chemistry reaction to form a triazole linkage between two probe oligonucleotides. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond.

In some embodiments, after ligation of the first and second probe oligonucleotides to create the ligated probe, the ligated probe is released from the analyte. In some embodiments, the ligated probe is released enzymatically. In some embodiments, an endoribonuclease is used to release the probe from the analyte. In some embodiments, the endoribonuclease is one or more of RNase H, RNase A, RNase C, or RNase I.

In some instances, the endoribonuclease is RNAse H. RNase H is an endoribonuclease that specifically hydrolyzes the phosphodiester bonds of RNA, when hybridized to DNA. RNase H is part of a conserved family of ribonucleases which are present in many different organisms. There are two primary classes of RNase H: RNase H1 and RNase H2. Retroviral RNase H enzymes are similar to the prokaryotic RNase H1. These enzymes share the characteristic that they are able to cleave the RNA component of an RNA:DNA heteroduplex. In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli.

In some embodiments, after creating a ligated probe from the probe oligonucleotides (e.g., a first probe oligonucleotide and second probe oligonucleotide), the biological sample is permeabilized. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin.

In some embodiments, the ligated probe includes a capture probe binding domain, which can hybridize to a capture probe (e.g., a capture probe immobilized, directly or indirectly, on a substrate). In some embodiments, methods provided herein include contacting a biological sample with a substrate, wherein the capture probe is affixed to the substrate (e.g., immobilized to the substrate, directly or indirectly). In some embodiments, the capture probe includes a spatial barcode and the capture domain. In some embodiments, the capture probe binding domain of the ligated probe specifically binds to the capture domain. After hybridization of the ligated probe to the capture probe, the ligated probe is extended at the 3′ end to make a copy of the additional components (e.g., the spatial barcode) of the capture probe.

In some embodiments, methods of ligated probe capture as provided herein include permeabilization of the biological sample such that the capture probe can more easily hybridize to the captured ligated probe (i.e., compared to no permcabilization).

The resulting products can be denatured from the capture probe template and transferred (e.g., to a clean tube) for amplification, and/or library construction as described herein. The spatially-barcoded, full-length probe product can be amplified via PCR prior to library construction. P5, P7, i7, and i5 can be incorporated into the library as for downstream sequencing. The fragments can then be sequenced using paired-end sequencing using TruSeq Read 1 and TruSeq Read 2 as sequencing primer sites. In some instances, the library is sequenced using any method described herein, such that different sequencing domains specific to other sequencing methods and techniques can be incorporated into a capture probe or introduced during library preparation. In some instances, the sequence of the templated ligation product is determined via sequencing. In some instances, the spatial barcode is sequenced, providing the location of the analyte.

Detailed descriptions of targeted RNA capture using templated ligation or RNA-templated ligation (RTL) has been disclosed in U.S. application No. 62/952,736, the entirety of which is incorporated herein by reference.

(c) SNP Detection using Templated Ligation

In some embodiments, provided herein are methods for determining the presence or absence of a genetic variant (e.g., a SNP) at a spatial location in a biological sample where (i) the first probe oligonucleotide and second probe oligonucleotide overlap to form an invasive cleavage structure in the presence of the target sequence, (ii) the formation of the invasive cleavage structure results in cleavage, and (iii) a ligated probe is generated. An invasive cleavage structure is described in U.S. Pat. No. 6,913,881 B1, which is incorporated by reference in its entirety. In some instances, because the probe oligonucleotides hybridize to immediately adjacent sequences, no enzyme set is necessary to extend either probe oligonucleotide.

In some embodiments, the methods of detecting a SNP using templated ligation uses two oligonucleotide probes that are hybridized to target nucleic acid (e.g., mRNA) containing a polymorphic site. The two oligonucleotides hybridize to the single-stranded target and form an overlapping invader structure at the site of the SNP. One oligonucleotide is complementary to the target sequence 3′ of the polymorphic site and ends with a non-matching base overlapping the SNP nucleotide. The second oligonucleotide, the allele-specific probe, contains the complementary base of the SNP allele and extends to the sequence 5′ of the polymorphic site. In some instances, this probe can also extend on its 5′ site with additional non-complementary nucleotides. Once the two oligonucleotides hybridize to the target nucleic acid (e.g., mRNA), they form a three-dimensional invader structure over the SNP site that can be recognized by a cleavase, such as a FEN enzyme. The enzyme cleaves the 5′ non-complementary nucleotides of the second probe oligonucleotide. Once cleaved, a free phosphate is created that allows a ligase described herein to ligate the two probe oligonucleotides. Additional exemplary support for the invasive cleavage structure utilized in an Invader assay is provided in Olivier, Mutat. Res. 2005 June 3; 573(1-2): 103-110, which is incorporated by reference in its entirety.

In one feature of the disclosure, the method for determining the presence or absence of a genetic variant in an analyte includes (a) contacting the biological sample with a first probe oligonucleotide (e.g., any of the first probe oligonucleotides described herein) and a second probe oligonucleotide (e.g., any of the second probe oligonucleotides described herein), where the first probe oligonucleotide and the second probe oligonucleotide each include a sequence that is substantially complementary to adjacent sequences of the analyte; where the first probe oligonucleotide and the second probe oligonucleotide are capable of forming an invasive cleavage structure in the presence of the genetic variant; and where: (i) the first probe oligonucleotide further includes at least one nucleotide that is complementary to a wild-type sequence of the genetic variant; and (ii) the second probe oligonucleotide further includes: a sequence of non-complementary nucleotides; a nucleotide complementary to the genetic variant; and a capture probe binding domain; (b) hybridizing the first probe oligonucleotide and the second probe oligonucleotide to the analyte; (c) cleaving the sequence of non-complementary nucleotides when the genetic variant is present; (d) ligating the first probe oligonucleotide and the second probe oligonucleotide, thereby creating a ligated probe; (e) releasing the ligated probe from the analyte; (f) contacting the biological sample with a substrate including a plurality of capture probes, where a capture probe of the plurality includes a spatial barcode and the capture domain; (g) hybridizing the ligated probe to the capture domain; and (h) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the genetic variant, if it is present, in the biological sample. In some embodiments, the method includes repeating steps (a)-(c) with a second set of first probe oligonucleotides and second set of second probe oligonucleotides.

In another feature of the disclosure, the method for determining the presence or absence of a genetic variant in an analyte includes (a) contacting the biological sample with a first probe oligonucleotide and a second probe oligonucleotide, where the first probe oligonucleotide and the second probe oligonucleotide each include a sequence that is substantially complementary to adjacent sequences of the analyte, where the first probe oligonucleotide and the second probe oligonucleotide are capable of forming an invasive cleavage structure in the presence of the analyte, where the second probe oligonucleotide includes a capture probe binding domain; (b) hybridizing the first probe oligonucleotide and the second probe oligonucleotide to the analyte; (c) ligating the first probe oligonucleotide and the second probe oligonucleotide, thereby creating a ligated probe; (d) releasing the ligated probe from the analyte; (c) contacting the biological sample with a substrate including a plurality of capture probes, where a capture probe of the plurality includes a spatial barcode and the capture domain; (f) hybridizing the ligated probe to the capture domain; and (g) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the analyte in the biological sample.

In some embodiments, the first probe oligonucleotide includes a sequence that is substantially complementary to a sequence 3′ of the target sequence (e.g., the genetic variant). In some embodiments, the first probe oligonucleotide is at least 70% identical (e.g., at least 75% identical, at least 80% identical, at least 85% identical, at least 90% identical, at least 95% identical, or at least 99% identical) to a sequence 3′ of the target sequence (e.g., the genetic variant). In some embodiments, the first probe oligonucleotide includes a sequence that is substantially complementary to a sequence 5′ of the target sequence (e.g., the genetic variant). In some embodiments, the first probe oligonucleotide is at least 70% identical (e.g., at least 75% identical, at least 80% identical, at least 85% identical, at least 90% identical, at least 95% identical, or at least 99% identical) to a sequence 5′ of the target sequence (e.g., the genetic variant). Thus, in some instances, because the first probe can be designed to hybridize to a genetic variant, it can be considered a “modified” first probe compared to the version of the second probe that hybridizes to a wild-type sequence.

In some embodiments, the first probe oligonucleotide includes at least one nucleotide that is complementary to a reference target sequence (e.g., wild type sequence of a genetic variant). In some embodiments, the first probe oligonucleotide includes at least one nucleotide that is complementary to a single nucleotide polymorphism (e.g., a single nucleotide polymorphism as compared to a reference target sequence).

In some embodiments, the second probe oligonucleotide includes a sequence of non-complementary nucleotides (e.g., a 5′ Flap). In some embodiments, non-complementary nucleotides include one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or at least 25 nucleotides. In some instances, the sequence of the 5′ Flap is a random sequence. In some instances, the sequence of the 5′ Flap is a sequence designed to hybridize to a different analyte of interest. In the instance in which the 5′ Flap is associated with (e.g., conjugated to) a detection moiety (e.g., a fluorescent moiety), the sequence of the 5′ Flap can be designed to hybridize to an analyte that allows for stable detection of the detection moiety. This form of detection serves as an alternative method of detection of probe hybridization. In some instances, the 5′ Flap does not include a detection moiety. In this instance, identification of the ligation product is performed via downstream analysis (e.g., sequencing the ligated probe or a complement thereof after hybridization to a probe on an array).

In some embodiments, the second probe oligonucleotide includes at least one nucleotide complementary to the at least one genetic variant of the target sequence. In some embodiments, the second probe oligonucleotide includes at least one nucleotide complementary to a reference target sequence (e.g., a wild type sequence).

In some embodiments, the second probe oligonucleotide includes a sequence substantially complementary to a sequence 5′ to the target sequence (e.g., the genetic variant). In some embodiments, the second probe oligonucleotide is at least 70% identical (e.g., at least 75% identical, at least 80% identical, at least 85% identical, at least 90% identical, at least 95% identical, or at least 99% identical) to a sequence 5′ of the target sequence (e.g., the genetic variant). In some embodiments, the second probe oligonucleotide includes a sequence substantially complementary to a sequence 3′ to the target sequence (e.g., the genetic variant). In some embodiments, the second probe oligonucleotide is at least 70% identical (e.g., at least 75% identical, at least 80% identical, at least 85% identical, at least 90% identical, at least 95% identical, or at least 99% identical) to a sequence 3′ of the target sequence (e.g., the genetic variant). Thus, in some instances, because the second probe can be designed to hybridize to a genetic variant, it can be considered a “modified” second probe compared to the version of the second probe that hybridizes to a wild-type sequence.

In some embodiments, the second probe oligonucleotide includes from 5′ to 3′: a sequence of non-complementary nucleotides, one or more nucleotides complementary to the target sequence, a sequence substantially complementary to a sequence 5′ of the target sequence (e.g., the genetic variant), and a capture probe binding domain (e.g., any of the capture probe binding domains described herein) that is capable of binding to a capture domain of a capture probe.

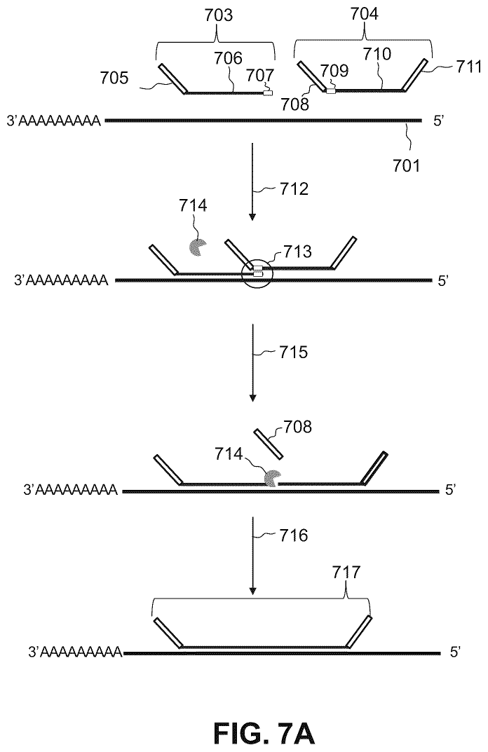

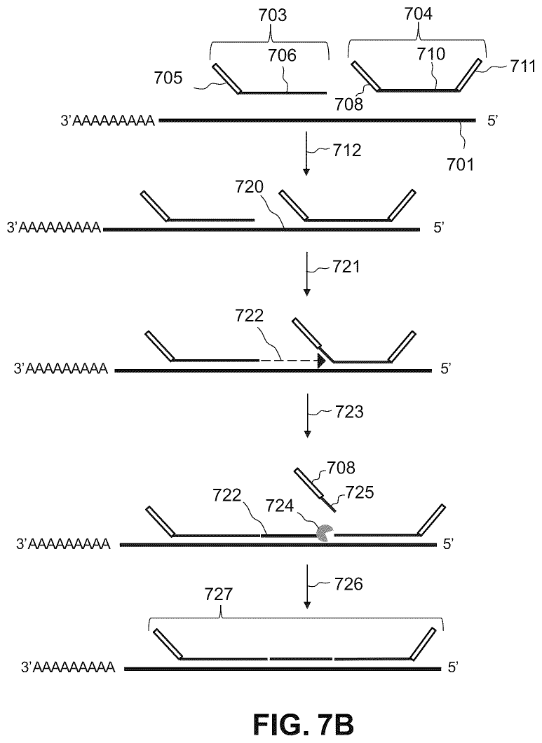

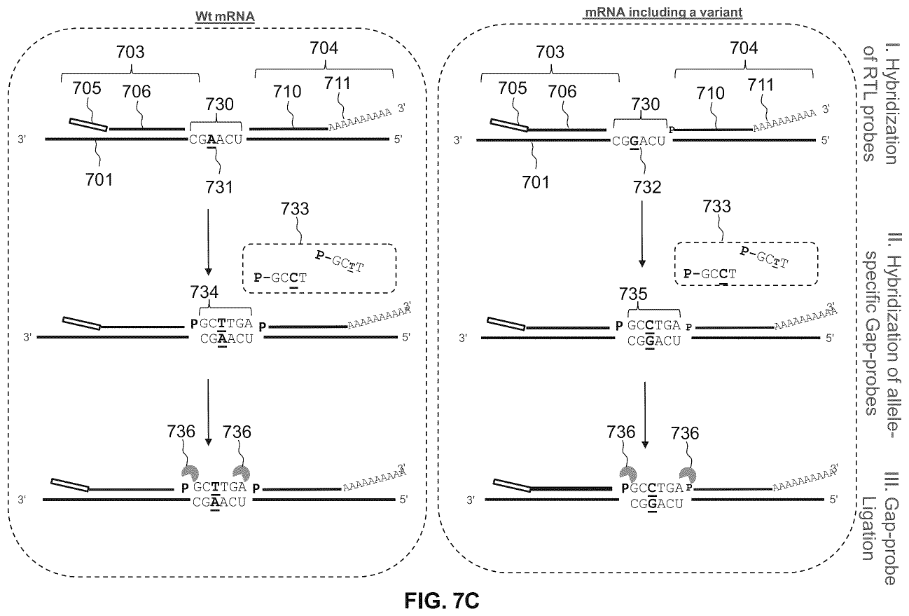

Referring to FIG. 7A, in some instances, the probes are designed so that the genetic variant to be detected is at the area where the two probes hybridize. In some instances, one probe 703 has a sequence 707 that hybridizes to the wild-type sequence. Then, a second probe 704 has a sequence 709 that hybridizes to a different (e.g., SNP) sequence. Thus, each probe competes to hybridize to the analyte at the position of the SNP, and only one probe hybridizes because it is complementary to the analyte at that position.

In some embodiments, the target sequence in an analyte of interest includes one or more nucleotides. In some embodiments, the target sequence includes one or more single nucleotide variants (SNV or SNP) compared to a reference target sequence. In some instances, the target sequence includes one SNV compared to a reference target sequence. In some embodiments, the target sequences include one or more genetic variants compared to a reference target sequence.

In some embodiments, after formation of the invader structure, an endonuclease is provided. In some instances, the endonuclease cleaves the sequence of non-complementary nucleotides (e.g., a 5′ Flap) of the second probe oligonucleotide. In some embodiments, the endonuclease cleaves a portion of the first probe oligonucleotide. In some embodiments, the endonuclease is Flap endonuclease 1 (FEN1). FEN1 is a structure-specific endonuclease that cuts at the base of single-stranded flaps. Sec Balakrishnan and Bambara, Annu Rev Biochem. 2013 Jun. 2; 82: 119-138, which is incorporated by reference in its entirety.

In some embodiments, the endonuclease cleaves the at least one nucleotide that is complementary to a wild-type sequence of the target sequence of the first probe oligonucleotide.

In some embodiments, the endonuclease cleaves a portion of the second probe oligonucleotide. In some embodiments, the endonuclease cleaves the sequence of non-complementary nucleotides of the second probe. In some embodiments, the non-complementary nucleotides include a sequence that is capable of binding to a second capture domain. For example, the non-complementary nucleotides of the second probe can include a second capture probe binding domain. In such cases, upon endonuclease-mediated cleavage of the non-complementary nucleotides from the second probe, the cleaved non-complementary nucleotides can bind a second capture domain. In some embodiments, binding of the cleaved non-complementary nucleotides to a second capture domain serves as the basis for determining the presence or absence of one or more genetic variants at the target sequence.

In some embodiments, the first probe oligonucleotide further includes a first moiety. In some embodiments, the first moiety is a first fluorophore (e.g., any of the exemplary fluorophores described herein). In some embodiments, the first fluorophore emits a first signal when the first probe oligonucleotide is cleaved.

In some embodiments, the second probe oligonucleotide further includes a second moiety. In some embodiments, the second moiety is a second fluorophore (e.g., any of the exemplary fluorophores described herein). In some embodiments, the second fluorophore emits a second signal when the second probe oligonucleotide is cleaved. In some embodiments, the first signal and the second signal are different. In some instances, the first signal is at a first wavelength and the second signal is at a second wavelength. In some instances, the wavelengths detect different fluorescent markers described herein.

In some instances, the second probe oligonucleotide is designed as a fluorescence resonance energy transfer (FRET) molecule containing a fluorophore at the 5′ end and an internal quencher molecule. In some instances, the cleavage reaction will separate the fluorophore from the quencher thereby allowing the fluorophore to generate a measurable fluorescent signal. In some instances, if the probe oligonucleotide does not match the SNP allele present in the target DNA (i.e. the probe is complementary to the alternate SNP allele), then no overlapping invader structure is formed, and the probe is not cleaved.

In some embodiments, the at least one genetic variant is a single nucleotide polymorphism (SNP). In some embodiments, the at least one genetic variant is a nucleotide point mutation. In some embodiments, the at least one genetic variant includes at least two, at least three, at least four, at least five, or more genetic variants.

In some instances, once FEN1 cleaves the 5′ Flap, the hybridized probe oligonucleotides are ligated using methods disclosed herein (e.g., enzymatically using e.g., T4 DNA ligase or chemically). In some embodiments, the ligase is a pre-activated T4 DNA ligase as described herein. A non-limiting example describing methods of generating and using pre-activated T4 DNA include U.S. Pat. No. 8,790,873, the entire contents of which are herein incorporated by reference. In some instances, a ligated product is formed, and the ligated product is cleaved from the analyte (e.g., mRNA) using an endoribonuclease. In some embodiments, the endoribonuclease is one or more of RNase H, RNase A, RNase C, or RNase I.

In some embodiments, after creating a ligated probe from the probe oligonucleotides (e.g., a first probe oligonucleotide and second probe oligonucleotide), the biological sample is permeabilized. In some embodiments, permeabilization occurs using a protease (e.g., an endopeptidase disclosed herein).

In some embodiments, the ligated probe includes a capture probe binding domain (e.g., which originated from the second probe), which can hybridize to a capture probe (e.g., a capture probe immobilized, directly or indirectly, on a substrate). In some embodiments, methods provided herein include contacting a biological sample with a substrate, where the capture probe is affixed to the substrate (e.g., immobilized to the substrate, directly or indirectly). In some embodiments, the capture probe includes a spatial barcode and the capture domain. In some embodiments, the capture probe binding domain of the ligated probe specifically binds to the capture domain. After hybridization of the ligated probe to the capture probe, the ligated probe is extended at the 3′ end to make a copy of the additional components (e.g., the spatial barcode) of the capture probe. In some embodiments, methods of ligated probe capture as provided herein include permeabilization of the biological sample such that the capture probe can more easily hybridize to the captured ligated probe (i.e., compared to no permeabilization). In some embodiments, reverse transcription (RT) reagents can be added to permeabilized biological samples. Incubation with the RT reagents can produce spatially-barcoded full-length ligation products from the captured analytes (e.g., polyadenylated mRNA).

The resulting extended ligation product can be denatured from the capture probe template and transferred (e.g., to a clean tube) for amplification, and/or library construction as described herein. The spatially-barcoded, full-length extended product can be amplified via PCR prior to library construction. The extended product can then be enzymatically fragmented and size-selected in order to optimize the amplicon size. In some instances, P5, P7, i7, and i5 can be incorporated into the library as for downstream sequencing, and additional library sequencing regions can be added via End Repair, A-tailing, Adaptor Ligation, and/or PCR. The fragments can then be sequenced using paired-end sequencing using TruSeq Read 1 and TruSeq Read 2 as sequencing primer sites. In some instances, the DNA library is sequenced using any method described herein, such that different sequencing domains specific to other sequencing methods and techniques can be incorporated into a capture probe or introduced during library preparation. In some instances, the sequence of the templated ligation product is determined via sequencing. In some instances, the spatial barcode is sequenced, providing the location of the analyte.

### (d) Templated Ligation and Gap Filling

In some embodiments, provided herein are methods for identifying a location of an analyte in a biological sample through a method that utilizes templated ligation of multiple (e.g., two) oligonucleotides that hybridize to substantially complementary sequences that are not immediately adjacent to one another. In some instances, there is a sequence in the analyte between the hybridized probes of at least 1-100, 1-90, 1-80, 1-70, 1-60, 1-50, 1-40, 1-30, 1-20, 1-10, 1-9, 1-8, 1-7, 1-6, 1-5, 1-4, 1-3, 1-2 or 1 nucleotide(s).

In some instances, the methods disclosed herein provide steps wherein only one set of probe oligonucleotides are needed around a mutation “hotspot” in an analyte (e.g., a target sequence). In a non-limiting example, a first probe oligonucleotide having a sequence that is complementary to a sequence 3′ of the target sequence and a second probe oligonucleotide having a sequence that is complementary to a sequence 5′ of the target sequence each bind to an analyte leaving a sequence (e.g., a “gap”) in between the first and second probes that is gap-filled thereby enabling ligation and generation of a ligated probe. In some instances, to generate probe oligonucleotides that are close enough to one another to initiate a ligation step, one or more of the probe oligonucleotides is extended enzymatically (e.g., using a polymerase).

In one feature of the disclosure, the method for identifying a location of an analyte in a biological sample using templated ligation and gap filling includes (a) contacting the biological sample with a first probe oligonucleotide (e.g., any of the first probe oligonucleotides described herein) and a second probe oligonucleotide (e.g., any of the second probe oligonucleotides described herein), where the first probe oligonucleotide includes a sequence substantially complementary to a sequence 3′ of a target sequence, the second probe oligonucleotide includes: a sequence of non-complementary nucleotides; a sequence substantially complementary to a sequence 5′ of the target sequence and a capture probe binding domain, and the target sequence is at least one nucleotide; (b) hybridizing the first probe oligonucleotide and the second probe oligonucleotide to the analyte; (c) extending the first probe oligonucleotide to generate an extended first probe oligonucleotide; (d) cleaving the sequence of non-complementary nucleotides; (e) ligating the extended first probe oligonucleotide and the second probe oligonucleotide, thereby creating a ligated probe that is substantially complementary to the analyte; (f) releasing the ligated probe from the analyte; (g) contacting the biological sample with a substrate, where the capture probe is affixed to the substrate, where the capture probe includes a spatial barcode and the capture domain; (h) allowing the capture probe binding domain of the ligated probe to specifically bind to the capture domain; and (i) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the location of the analyte in the biological sample.

In some embodiments, the target “gap” sequence between the hybridized probe oligonucleotides includes one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides.

In some embodiments, the complementary sequences to which the first probe oligonucleotide and the second probe oligonucleotide bind are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other.

In some embodiments, the second probe oligonucleotide includes from 5′ to 3′: a sequence of non-complementary nucleotides, a sequence substantially complementary to a sequence 5′ of the target sequence and a capture probe binding domain (e.g., any of the capture probe binding domains described herein) that is capable of binding to a capture domain of a capture probe.

In some embodiments, the second probe oligonucleotide includes a sequence of non-complementary nucleotides. In some instances, the sequence of non-complementary nucleotides is at the 5′ end of the second probe nucleotide, creating a 5′ Flap. In some embodiments, the sequence of non-complementary nucleotides includes one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or at least 25 nucleotides. In some instances, the sequence of the 5′ Flap is a random sequence. In some instances, the sequence of the 5′ Flap is a sequence designed to hybridize to a different analyte of interest. In the instance in which the 5′ Flap is associated with (e.g., conjugated to) a detection moiety (e.g., a fluorescent moiety), the sequence of the 5′ Flap can be designed to hybridize to an analyte that will allow for stable detection of the detection moiety. This form of detection serves as an alternative method of detection of probe hybridization. In some instances, the 5′ Flap does not include a detection moiety. In this instance, identification of the ligation product is performed via downstream analysis (e.g., sequencing the ligated probe or a complement thereof after hybridization to a probe on an array).

In some instances, the second probe oligonucleotide does not include a 5′ Flap.

In some embodiments, extending the first probe oligonucleotide includes a nucleic acid extension reaction (e.g., any of the nucleic acid extension reactions described herein). In some embodiments, after cleaving the 5′ Flap, extending the first probe oligonucleotide includes reverse transcribing the target sequence. In some embodiments, extending the first probe oligonucleotide includes using a reverse transcriptase (e.g., any of the reverse transcriptases described herein). In some embodiments, extending the first probe oligonucleotide includes using a Moloney Murine Leukemia Virus (M-MulV) reverse transcriptase. In some embodiments, any enzyme that can extend a nucleic acid strand and exhibits strand displacement properties can be used to extend the first probe oligonucleotide. In some embodiments, extending the first probe oligonucleotide generates a sequence that is complementary to the target sequence. In some embodiments, extending the first probe oligonucleotide generates an extended first probe oligonucleotide that is complementary to the target sequence.

In some embodiments, extending the first probe oligonucleotide generates a sequence that is adjacent to the second probe oligonucleotide.

In some embodiments, extending the first probe oligonucleotide creates an invasive cleavage structure in which the 5′ Flap of the second probe oligonucleotide is cleaved. See Olivier, Mutat. Res. 2005 June 3; 573(1-2): 103-110, which is incorporated by reference in its entirety. In some instance, at least part of the second probe oligonucleotide is displaced. When part of the second probe oligonucleotide is displaced, endonuclease cleavage (e.g., cleavage using any of the endonucleases described in herein) generates probes that can be ligated. In such cases, as described above, a FEN1 endonuclease can be used for cleavage.

In some embodiments, the ligation step includes ligating the extended first probe oligonucleotide and the second probe oligonucleotide using enzymatic or chemical ligation. In some embodiments where the ligation is enzymatic, the ligase is selected from a T4 RNA ligase (Rnl2), a splintR ligase, a single stranded DNA ligase, or a T4 DNA ligase. In some embodiments, the ligase is a T4 RNA ligase (Rnl2) ligase. In some embodiments, the ligase is a pre-activated T4 DNA ligase as described herein. A non-limiting example describing methods of generating and using pre-activated T4 DNA include U.S. Pat. No. 8,790,873, the entire contents of which are herein incorporated by reference.

In some features of this disclosure, provided herein are methods for identifying a location of an analyte in a biological sample using templated ligation and gap filling includes (a) contacting a biological sample with a first probe oligonucleotide and a second probe oligonucleotide, where the first probe oligonucleotide includes a sequence substantially complementary a sequence 3′ of a target sequence, where the second probe oligonucleotide includes a sequence of non-complementary nucleotides, a sequence substantially complementary to a sequence 5′ of the target sequence and a capture probe binding domain that is capable of binding to a capture domain of a capture probe, and where the complementary sequences to which the first probe oligonucleotide and the second probe oligonucleotide bind are more than one nucleotide away from each other; (b) hybridizing the first probe oligonucleotide and the second probe oligonucleotide to the analyte; (c) extending the first probe oligonucleotide to generate an extended first probe oligonucleotide; (d) cleaving the sequence of non-complementary nucleotides of the second probe (e) ligating the extended first probe oligonucleotide and the second probe oligonucleotide, thereby creating a ligated probe that is substantially complementary to the analyte; (f) releasing the ligated probe from the analyte; (g) contacting the biological sample with a substrate, where the capture probe is affixed to the substrate, where the capture probe includes a spatial barcode and the capture domain; (h) allowing the capture probe binding domain of the ligated probe to specifically bind to the capture domain; and (i) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the location of the analyte in the biological sample.

In some embodiments, the cleaving step includes an endonuclease (e.g., any of the endonucleases described herein). In some embodiments, the endonuclease is Flap endonuclease 1 (FEN1). In some embodiments, the endonuclease is selected from a Thermus aquaticus (Taq) DNA polymerase, a Thermus thermophilus (Tth) polymerase, Xeroderma pigmentosum group G (XPG) protein, Rad2 from budding yeast, AtRAD2 from Arabidopsis, or Rad13 in fission yeast. In some embodiments, the endonuclease is Thermus aquaticus (Taq) DNA polymerase. In some instances, the endonuclease is a Thermus thermophilus (Tth) polymerase. In some instances, the endonuclease is selected from RAD2, an XPG (Xeroderma Pigmentosa-complementation group G) protein, E. coli DNA polymerase, or the archeal or eukaryotic flap endonuclease 1 (FEN1), including murine FEN1, yeast FEN1, human FEN1, P. horikoshii Pho FEN1, Pfu FEN1, and derivatives thereof. In some instances, the endonuclease is selected from the group consisting of I-Anil, I-Scel, I-Ceul, PI-PspI, PI-Sce, I-SceIV, I-CsmI, I-PanI, I-PanII, I-PanMI, I-Scell, I-Ppol, I-SceIII, I-Crel, I-LtrI, I-Gpil, I-GZel, I-Onul, I-HjeMI, I-TevI, I-TevII, and I-TevIII with one or more DNA end-processing enzymes selected from the group consisting of: Trex2, Trex1, Trex1 without transmembrane domain, Apollo, Artemis, DNA2, Fan1, MreII, Rad2, Rad9, TdT (terminal deoxynucleotidyl transferase), PNKP, RecE, RecJ, RecQ, Sox, Vaccinia DNA polymerase, NDK1, NDK5, NDK7, NDK8, WRN, avian myeloblastosis virus integration protein (IN), Bloom, Antartic Phophatase, Alkaline Phosphatase, Poly nucleotide Kinase (PNK), Apel, Mung Bean nuclease, Hex1, TTRAP (TDP2), Sgs1, Sae2, CtIP, Pol mu, Pol lambda, MUS81, EME1, EME2, SLX1, SLX4 and UL-12, or derivatives thereof.

In some embodiments, the endonuclease cleaves at least the non-complementary nucleotides from the second probe oligonucleotide. In some embodiments where extending the first probe oligonucleotide results in strand displacement of part of the second probe oligonucleotide, the endonuclease cleaves the part of the second probe oligonucleotide not bound to the target sequence. In some embodiments, the non-complementary nucleotides include a sequence that is capable of binding to a second capture domain. For example, the non-complementary nucleotides of the second probe can include a second capture probe binding domain.

After a ligation product is created, downstream applications that facilitate identification of the analyte or ligation product in a spatial context can be performed. For example, in some instances, after a ligated product is formed, the ligated product is released from the analyte (e.g., mRNA) using an endoribonuclease. In some embodiments, the endoribonuclease is one or more of RNase H, RNase A, RNase C, or RNase I. In some embodiments, after creating a ligated probe from the probe oligonucleotides (e.g., a first probe oligonucleotide and second probe oligonucleotide), the biological sample is permeabilized. In some embodiments, permeabilization occurs using a protease (e.g., an endopeptidase disclosed herein).

In some embodiments, the ligated probe includes a capture probe binding domain, which can hybridize to a capture probe (e.g., a capture probe immobilized, directly or indirectly, on a substrate). In some embodiments, methods provided herein include contacting a biological sample with a substrate, where the capture probe is affixed to the substrate (e.g., immobilized to the substrate, directly or indirectly). In some embodiments, the capture probe includes a spatial barcode and the capture domain. In some embodiments, the capture probe binding domain of the ligated probe specifically binds to the capture domain. After hybridization of the ligated probe to the capture probe, the ligated probe is extended at the 3′ end to make a copy of the additional components (e.g., the spatial barcode) of the capture probe. In some embodiments, methods of ligated probe capture as provided herein include permeabilization of the biological sample such that the capture probe can more easily hybridize to the captured ligated probe (i.e., compared to no permeabilization). In some embodiments, reverse transcription (RT) reagents can be added to permeabilized biological samples. Incubation with the RT reagents can produce spatially-barcoded full-length ligation products from the.

The resulting extended products can be denatured from the capture probe template and transferred (e.g., to a clean tube) for amplification, and/or library construction as described herein. The spatially-barcoded, full-length extension products can be amplified via PCR prior to library construction. The products can be enzymatically fragmented and size-selected in order to optimize the amplicon size. P5, P7, i7, and i5 can be incorporated into the library as for downstream sequencing, and additional library sequencing regions can be added via End Repair, A-tailing, Adaptor Ligation, and/or PCR. The fragments can then be sequenced using paired-end sequencing using TruSeq Read 1 and TruSeq Read 2 as sequencing primer sites. In some instances, the library is sequenced using any method described herein, such that different sequencing domains specific to other sequencing methods and techniques can be incorporated into a capture probe or introduced during library preparation. In some instances, the sequence of the templated ligation product is determined via sequencing. In some instances, the spatial barcode is sequenced, providing the location of the analyte.

### (c) Templated Ligation Using Gap Probes

In some embodiments provided herein are methods for identifying a location of an analyte having one or more genetic variants in a biological sample using a third oligonucleotide. Similar to the disclosure in section (VI)(c) above, a gap is created between the two probe oligonucleotides upon hybridization to the analyte. In some instances, a third probe is included that is complementary to the gap sequence between the hybridized probe oligonucleotides. In some instances, the gap sequence includes one or more mutations (e.g., in a mutational hotspot region of an analyte). Designing probes that are complementary to the particular sequence intervening the two hybridized probe oligonucleotides increases efficiency of (1) hybridization of the third probe to the gap sequence and (2) post-hybridization ligation. In addition, the methods disclosed herein provide increased ligation efficiency and detection of one or more mutations in an intervening sequence as ligases possess poor fidelity at discriminating mismatches at a ligation site. In a non-limiting example, a first probe oligonucleotide having a sequence that is substantially complementary to a sequence 3′ of the target sequence, a second probe oligonucleotide having a sequence that is substantially complementary to a sequence 5′ of the target sequence, and a gap probe oligonucleotide are used to determine the location of an analyte in a biological sample.

In one feature of the disclosure, provided herein is a method for identifying a location of an analyte having one or more genetic variants in a biological sample including (a) contacting the biological sample with a first probe oligonucleotide (e.g., any of the first probe oligonucleotides described herein) and a second probe oligonucleotide (e.g., any of the second probe oligonucleotides described herein), where the first probe oligonucleotide includes a sequence substantially complementary to a sequence 3′ of a target sequence, where the second probe oligonucleotide includes a sequence substantially complementary to a sequence 5′ of the target sequence and a capture probe binding domain that is capable of binding to a capture domain of a capture probe, and where the complementary sequences to which the first probe oligonucleotide and the second probe oligonucleotide bind are more than one nucleotide away from each other; (b) hybridizing the first probe oligonucleotide and the second probe oligonucleotide to the analyte; (c) contacting the biological sample with a plurality of gap probe oligonucleotides, where a gap probe oligonucleotide of the plurality of gap probe oligonucleotides includes a sequence substantially complementary to the target sequence; (d) ligating (i) the first probe oligonucleotide and the 5′ end of a gap probe oligonucleotide and (ii) the second probe oligonucleotide and the 3′ end of the gap probe oligonucleotide, thereby creating a ligated probe that is substantially complementary to the analyte; (c) releasing the ligated probe from the analyte; (f) contacting the biological sample with a substrate including a plurality of capture probes, where a capture probe of the plurality includes a spatial barcode and the capture domain; (g) hybridizing the ligated probe to the capture domain; and (h) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the analyte in the biological sample. In some instances, the steps of hybridizing (step (b)) and contacting (step (c)) occur at substantially the same time (i.e., in a single step). In some instances, the steps of hybridizing (step (b)) and contacting (step (c)) occur at substantially at different times (i.e., in multiple steps).

In some embodiments, the method includes contacting the biological sample with a plurality of gap probe oligonucleotides (e.g., any of the gap probe oligonucleotides described herein) prior to contacting the biological sample with a first probe oligonucleotide (e.g., any of the first probe oligonucleotides described herein) and a second probe oligonucleotide (e.g., any of the second probe oligonucleotide described herein). In some instances, a cocktail of gap probe oligonucleotides is introduced to the biological sample. In some instances, each gap probe oligonucleotide is designed to be sequence-specific. In some instances, the sequences of the gap probe oligonucleotide include wild-type and/or mutation sequences. For example, in a setting where there are two known mutations (m1 and m2) in the analyte sequence between the hybridized probe oligonucleotides, four different probes can be designed: probes comprising (1) only m1, (2) only m2, (3) both m1 and m2, and (4) neither m1 nor m2. In some instances, the probes compete to bind to the gap sequence and the sequence that is most complementary hybridizes to the gap sequence more efficiently than the sequences that are less complementary.

In some embodiments, the gap sequence (e.g., the target sequence) includes two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides. In some embodiments, the gap sequence is about 5 to 10 nucleotides. In some embodiments, the gap sequence (e.g., the target sequence) includes one or more single nucleotide variants compared to a reference target sequence. In some embodiments, the target sequence includes two, three, four, five, six, seven, eight, nine or ten or more single nucleotide variants compared to a reference target sequence. In some embodiments, the target sequence includes a mutational hotspot (e.g., two or more nucleotide variants compared to a reference target sequence). In some embodiments, the complementary sequences of the first probe oligonucleotide and the second probe oligonucleotide are n nucleotides (e.g., six nucleotides) away from each other (e.g., a six nucleotide gap). In some embodiments, this gap (e.g., gap of six nucleotides) includes the target sequence including a genetic variant as compared to a target reference sequence. In some instances, the gap probe oligonucleotide includes one or more modified bases. In some instances, the modified bases increase stability of the gap probe oligonucleotide.

In some embodiments, a gap probe oligonucleotide includes two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides. In some embodiments, the gap probe oligonucleotide includes a sequence that is substantially complementary to the target sequence. In some embodiments, the gap probe oligonucleotide is at least 70% identical (e.g., at least 75% identical, at least 80% identical, at least 85% identical, at least 90% identical, at least 95% identical, or at least 99% identical) to the target sequence. In some instances, the gap probe oligonucleotide is 100% complementary to the target sequence.

In some embodiments, the plurality of gap probe oligonucleotides (e.g., cocktail) includes a first gap probe oligonucleotide including a sequence complementary to a target sequence including one or more single nucleotide variants. In some embodiments, the plurality of gap probe oligonucleotides includes a second gap probe oligonucleotide including a sequence complementary to a reference target sequence. In some embodiments, the plurality of gap probe oligonucleotides includes one or more additional gap probe oligonucleotides including a sequence that is not complementary to the target sequence.

In some embodiments, the plurality of gap probe oligonucleotides includes a gap probe oligonucleotide that includes a sequence complementary to a target sequence including a nucleotide variant and a second gap probe oligonucleotide that includes a sequence complementary to a reference target sequence. In some embodiments, the plurality of gap probe oligonucleotides includes a gap probe oligonucleotide that includes a sequence complementary to a target sequence including a first nucleotide variant, a second gap probe oligonucleotide that includes a sequence complementary to a first reference target sequence, a third gap probe oligonucleotide that includes a sequence complementary to a second nucleotide variant, and a fourth gap probe oligonucleotide that includes a sequence complementary to a second reference target sequence.

Once the probe oligonucleotides and the gap probe are hybridized to the oligonucleotide of interest, ligase joins the adjacent probe sequences. In some embodiments, the ligation step includes ligating the first probe oligonucleotide and the 3′ end of a gap probe oligonucleotide using enzymatic or chemical ligation. In some embodiments, the ligation step includes ligating the second probe oligonucleotide and the 5′ end of a gap probe oligonucleotide using enzymatic or chemical ligation. In some embodiments where the ligation is enzymatic, the ligase is selected from a T4 RNA ligase (Rn12), a splintR ligase, a single stranded DNA ligase, or a T4 DNA ligase. In some embodiments, the ligase is a T4 RNA ligase 2 (Rn12) ligase. In some embodiments, the ligase is a pre-activated T4 DNA ligase. A non-limiting example describing methods of generating and using pre-activated T4 DNA include U.S. Pat. No. 8,790,873, the entire contents of which are herein incorporated by reference.

After a ligation product is created, downstream applications that facilitate identification of the analyte or ligation product in a spatial context can be performed. For example, in some instances, after a ligated product is formed, the ligated product is released from the analyte (e.g., mRNA) using an endoribonuclease. In some embodiments, the endoribonuclease is one or more of RNase H, RNase A, RNase C, or RNase I. In some embodiments, after creating a ligated probe from the probe oligonucleotides (e.g., a first probe oligonucleotide and second probe oligonucleotide), the biological sample is permeabilized. In some embodiments, permeabilization occurs using a protease (e.g., an endopeptidase disclosed herein).

In some embodiments, the ligated probe includes a capture probe binding domain, which can hybridize to a capture probe (e.g., a capture probe immobilized, directly or indirectly, on a substrate). In some embodiments, methods provided herein include contacting a biological sample with a substrate, where the capture probe is affixed to the substrate (e.g., immobilized to the substrate, directly or indirectly). In some embodiments, the capture probe includes a spatial barcode and the capture domain. In some embodiments, the capture probe binding domain of the ligated probe specifically binds to the capture domain. After hybridization of the ligated probe to the capture probe, the ligated probe is extended at the 3′ end to make a copy of the additional components (e.g., the spatial barcode) of the capture probe. In some embodiments, methods of ligated probe capture as provided herein include permeabilization of the biological sample such that the capture probe can more easily hybridize to the captured ligated probe (i.e., compared to no permeabilization). In some embodiments, reverse transcription (RT) reagents can be added to permeabilized biological samples. Incubation with the RT reagents can produce spatially-barcoded full-length ligation product.

The resulting extended ligation products can be denatured from the capture probe template and transferred (e.g., to a clean tube) for amplification, and/or library construction as described herein. The spatially-barcoded, full-length ligation product can be amplified via PCR prior to library construction. The products can be enzymatically fragmented and size-selected to optimize the amplicon size. P5, P7, i7, and i5 can be incorporated into the library as for downstream sequencing, and additional library sequencing regions can be added via End Repair, A-tailing, Adaptor Ligation, and/or PCR. The fragments can be sequenced using paired-end sequencing using TruSeq Read 1 and TruSeq Read 2 as sequencing primer sites. In some instances, the library is sequenced using any method described herein, such that different sequencing domains specific to other sequencing methods and techniques can be incorporated into a capture probe or introduced during library preparation. In some instances, the sequence of the templated ligation product is determined via sequencing. In some instances, the spatial barcode is sequenced, providing the location of the analyte.

### (f) Pre-Hybridization Methods

### (i) Imaging and Staining

Prior to addition of the probes (e.g., templated ligation probes), in some instances, biological samples can be stained using a wide variety of stains and staining techniques. In some instances, the biological sample is a section on a slide (e.g., a 5 μm section, a 7 μm section, a 10 μm section, etc.). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassic blue, cresyl violet, DAPI, cosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and cosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), cascin, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### (ii) Preparation of a Sample for Application of Probes

In some instances, the biological sample is deparaffinized. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° ° C. to about 80° ° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for probe application include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some instances, the methods of preparing a biological sample for probe application include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### (g) Hybridizing the Probes

In some embodiments, the methods described herein include hybridizing with a first probe oligonucleotide and/or a second probe oligonucleotide (e.g., a probe pair) for targeted RNA capture. In some instances, the first and second probe oligonucleotides for targeted RNA capture each include sequences that are substantially complementary to one or more sequences (e.g., one or more target sequences) of an analyte of interest. In some embodiments, only a part of each of the probe oligonucleotides hybridizes to the analyte, while the remaining sequences are not complementary to an analyte. In other words, in some embodiments, the first probe and the second probe each comprise (A) regions that are complementary to adjacent sequences on an analyte and (B) sequences that are not complementary to the analyte. Non-complementary sequences include primer sequences such as a Read 1 sequence, a 5′Flap region, and a capture binding sequence such as a poly(A) sequence or tail. For instance, in one embodiment, one probe comprises a primer sequence (e.g., a Read 1 sequence), which is not complementary to the analyte. In one embodiment, one probe comprises the primer sequence and the 5′Flap region. In some instances, one probe comprises a poly(A) tail.

In some instances, the methods include hybridization of probe sets in order to detect variants, wherein the probe pairs are in a medium at a concentration of about 1 to about 100 nM. In some instances, the concentration of the probe pairs is about 1, 2, 3, 4, 5, 6, 7, 8, 9, 10, 15, 20, 25, 30, 35, 40, 45, 50, 60, 70, 80, 90, 100, 200, 300, 400, or 500 nM. In some instances, the concentration of the probe pairs is 5 nM. In some instances, the probe sets are diluted in a hybridization (Hyb) buffer. In some instances, the probe sets are at a concentration of 5 nM in Hyb buffer.

In some instances, probe hybridization (e.g., the first and second probe oligonucleotides) to wild-type or variant sequences occurs at about 50° C. In some instances, the temperature of probe hybridization ranges from about 30° C. to about 75° C., from about 35° ° C. to about 70° C., or from about 40° ° C. to about 65° C. In some embodiments, the temperature is about 30° C., about 31° C., about 32° ° C., about 33° C., about 34° C., about 35° C., about 36° C., about 37° C., about 38° C., about 39° C., about 40° C., about 41° ° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° ° C., about 61° C., about 62° C., about 63° C., about 64° C., about 65° C., about 66° C., about 67° C., about 68° C., about 69° C., or about 70° ° C. In some instances, probe hybridization occurs for about 30 minutes, about 1 hour, about 2 hours, about 2.5 hours, about 3 hours, or more. In some instances, probe hybridization occurs for about 2.5 hours at 50° C.

In some instances, the hybridization buffer includes SSC (e.g., 1×SSC) or SSPE. In some instances, the hybridization buffer includes formamide or ethylene carbonate. In some instances, the hybridization buffer includes one or more salts, like Mg salt for example MgCl₂, Na salt for example NaCl, Mn salt for example MnCl₂. In some instances, the hybridization buffer includes Denhardt's solution, dextran sulfate, ficoll, PEG or other hybridization rate accelerators. In some instances, the hybridization buffer includes a carrier such as yeast tRNA, salmon sperm DNA, and/or lambda phage DNA. In some instances, the hybridization buffer includes one or more blockers. In some instances, the hybridization buffer includes RNase inhibitor(s). In some instances, the hybridization buffer can include BSA, sequence specific blockers, non-specific blockers, EDTA, RNase inhibitor(s), betaine, TMAC, or DMSO. In some instances, a hybridization buffer can further include detergents such as Tween, Triton-X 100, sarkosyl, and SDS. In some instances, the hybridization buffer includes nuclease-free water, DEPC water.

In some embodiments, the complementary sequences to which the first probe oligonucleotide and the second probe oligonucleotide bind are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides away from each other. Gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, when the first and second probe oligonucleotides are separated from each other by one or more nucleotides, nucleotides are ligated between the first and second probe oligonucleotides. In some embodiments, when the first and second probe oligonucleotides are separated from each other by one or more nucleotides, deoxyribonucleotides are ligated between the first and second probe oligonucleotides.

In some instances, after hybridization, the biological sample is washed with a post-hybridization wash buffer. In some instances, the post-hybridization wash buffer includes one or more of SSC, yeast tRNA, formamide, ethylene carbonate, and nuclease-free water.

Additional embodiments regarding probe hybridization are further provided.

### (i) Hybridizing Temperatures

In some embodiments, the method described herein utilizes oligonucleotides that can detect genetic variants and include deoxyribonucleic acids (instead of strictly utilizing ribonucleotides) at the site of ligation. Utilizing deoxyribonucleic acids in the methods described herein create a more uniform efficiency that can be readily-controlled and flexible for various applications. In some embodiments, a first probe oligonucleotide and/or a second probe oligonucleotide include deoxyribonucleic acids (instead of strictly utilizing ribonucleotides) at the site of ligation.

In a non-limiting example, the methods disclosed herein include contacting a biological sample with a plurality of oligonucleotides (e.g., modified templated ligation probes) in order to detect one or more genetic variants. In some instances, the methods include contacting the biological sample with a first oligonucleotide (e.g., a first probe) and a second oligonucleotide (e.g., a second probe), wherein the first oligonucleotide (e.g., the first probe) and the second oligonucleotide (e.g., the second probe) are complementary to a first sequence present in an analyte and a second sequence present in the analyte, respectively. In some instances, either the first sequence or the second sequence includes a genetic variant, and thus the first and/or second probes are designed to hybridize to the genetic variant. In some instances, the genetic variant is in between the two probe-binding sequences. Then in some instances, the methods further include hybridizing the first oligonucleotide (e.g., the first probe), and the second oligonucleotide (e.g., the second probe) to the analyte at a first temperature; hybridizing the first oligonucleotide (e.g., the first probe) and the second oligonucleotide (e.g., the second probe) to a third oligonucleotide (e.g., a splint oligonucleotide) at a second temperature such that the first oligonucleotide (e.g., the first probe) and the second oligonucleotide (e.g., the second probe) abut each other; ligating the first oligonucleotide (e.g., the first probe) to the second oligonucleotide (e.g., the second probe) to create a ligation product; contacting the biological sample with a substrate, wherein a capture probe is immobilized on the substrate, wherein the capture probe includes a spatial barcode and a capture domain; allowing the ligation product to specifically bind to the capture domain; and determining (i) all or a part of the sequence of the ligation product specifically bound to the capture domain, or a complement thereof, and (ii) all or a part of the sequence of the spatial barcode, or a complement thereof, and using the determined sequence of (i) and (ii) to identify the location of the analyte in the biological sample; wherein the first oligonucleotide (e.g., the first probe), the second oligonucleotide (e.g., the second probe), and the third oligonucleotide are DNA oligonucleotides, and wherein the first temperature is a higher temperature than the second temperature.

In some embodiments, the first oligonucleotide (e.g., the first probe) and/or the second oligonucleotide (e.g., the second probe) hybridize to an analyte at a first temperature. In some embodiments, the first temperature ranges from about 50° C. to about 75° C., from about 55° C. to about 70° C., or from about 60° C. to about 65° C. In some embodiments, the first temperature is about 55° C., about 56° C., about 57° C., about 58° C., about 59° C., about 60° C., about 61° C., about 62° C., about 63° C., about 64° C., about 65° C., about 66° ° C., about 67° C., about 68° C., about 69° ° C., or about 70° ° C.

In some embodiments, after the step of hybridizing the first oligonucleotide (e.g., the first probe) and/or the second oligonucleotide (e.g., the second probe) to the analyte, a wash step is performed to remove unbound oligonucleotides (e.g., probes). The wash step can be performed using any of the wash methods and solutions described herein.

In some embodiments, after the step of hybridizing the first oligonucleotide (e.g., the first probe) and the second oligonucleotide (e.g., the second probe) to the analyte, a third oligonucleotide (e.g., a splint oligonucleotide) is added to the analyte. In some embodiments, the third oligonucleotide is an oligonucleotide. In some embodiments, the third oligonucleotide is a DNA oligonucleotide.

In some embodiments, the third oligonucleotide includes a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a portion of the first probe oligonucleotide (e.g., a portion of the first probe that is not hybridized to the analyte (e.g., an auxiliary sequence)). In some embodiments, the third oligonucleotide includes a sequence that is 100% complementary to a portion of the first oligonucleotide (e.g., the first probe). In some embodiments, the third oligonucleotide includes a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a portion of the second probe oligonucleotide (e.g., a portion of the second probe that is not hybridized to the analyte (e.g., an auxiliary sequence)). In some embodiments, the third oligonucleotide includes a sequence that is 100% complementary to a portion of the second oligonucleotide (e.g., the second probe). In some embodiments, the third oligonucleotide hybridizes to the first oligonucleotide (e.g., the first probe) at the complementary portion. In some embodiments, the third oligonucleotide hybridizes to the second oligonucleotide (e.g., the second probe) at the complementary portion.

In some embodiments, the third oligonucleotide hybridizes to the first oligonucleotide (e.g., the first probe) and to the second oligonucleotide (e.g., the second probe) at a second temperature. In some embodiments, the second temperature is lower than the first temperature at which the first and second oligonucleotides (e.g., the first and second probes) bind the analyte. In some embodiments, the second temperature ranges from about 15° C. to about 35° C., from about 20° C. to about 30° ° C., or from about 25° C. to about 30° ° C. In some embodiments, the first temperature is about 15° C., about 16° C., about 17° C., about 18° C., about 19° C., about 20° C., about 21° C., about 22° ° C., about 23° C., about 24° C., about 25° C., about 26° C., about 27° C., about 28° C., about 29° C., about 30° ° C., about 31° C., about 32° C., about 33° C., about 34° C., or about 35° C. Methods including a third, or splint, oligonucleotide have been described in U.S. Patent Pub. No. 2019/0055594A1, which is herein incorporated by reference in its entirety.

In some embodiments, after the step of hybridizing the third oligonucleotide to the analyte, a wash step is performed to remove unbound third oligonucleotides. The wash step can be performed using any of the wash methods and solutions described herein. In some embodiments, after the washing step, the first and second oligonucleotides (e.g., the first and second probes) are bound to (e.g., hybridized to) the analyte, and the third oligonucleotide is bound to (e.g., hybridized to) the first and second oligonucleotides (e.g., at portions of the first and second probes that are not bound to the analyte).

In some embodiments, the first oligonucleotide (e.g., the first probe), the second oligonucleotide (e.g., the second probe), and the third oligonucleotide are added to the biological sample at the same time. Then, in some embodiments, the temperature is adjusted to the first temperature to allow the first oligonucleotide (e.g., the first probe) and the second oligonucleotide (e.g., the second probe) to hybridize to the analyte in the biological sample. Next, the temperature is adjusted to the second temperature to allow the third oligonucleotide to hybridize to the first oligonucleotide and the second oligonucleotide.

In some embodiments where a third oligonucleotide hybridizes to a first probe and a second probe that are hybridized to targets sequences that are not directly adjacent in the analyte, the third oligonucleotide is extended to fill the gap between the first probe and the second probe. In some instances, a polymerase (e.g., a DNA polymerase) can extend one of the probes (e.g., the first probe) prior to ligation.

In some embodiments, a ligation step is performed. Ligation can be performed using any of the methods described herein. In some embodiments, the step includes ligation of the first oligonucleotide (e.g., the first probe) and the second oligonucleotide (e.g., the second probe), forming a ligation product. In some embodiments, the third oligonucleotide serves as an oligonucleotide splint to facilitate ligation of the first oligonucleotide (e.g., the first probe) and the second oligonucleotide (e.g., the second probe). In some embodiments, ligation is chemical ligation. In some embodiments, ligation is enzymatic ligation. In some embodiments, the ligase is a T4 RNA ligase (Rnl2), a splintR ligase, a single stranded DNA ligase, or a T4 DNA ligase.

### (ii) Hybridization Buffer

In some embodiments, a first probe, and/or a second probe are hybridized to the analyte in order to detect one or more variants in a hybridization buffer. In some instances, the hybridization buffer contains formamide. In other instances the hybridization buffer is formamide frec. Formamide is not human friendly and it is a known health hazard. Chemically, it can oxidize over time, thereby impacting reagent shelf life and, most importantly, reagent efficacy. As such, the methods described herein can include formamide-free buffers, including formamide-free hybridization buffer.

In some embodiments, the formamide-free hybridization buffer is a saline-sodium citrate (SSC) hybridization buffer. In some embodiment, the SSC is present in the SSC hybridization buffer from about 1×SSC to about 6×SSC (e.g., about 1×SSC to about 5×SSC, about 1×SSC to about 4×SSC, about 1×SSC to about 3×SSC, about 1×SSC to about 2×SSC, about 2×SSC to about 6×SSC, about 2×SSC to about 5×SSC, about 2×SSC to about 4×SSC, about 2×SSC to about 3×SSC, about 3×SSC to about 5×SSC, about 3×SSC to about 4×SSC, about 4×SSC to about 6×SSC, about 4×SSC to about 6×SSC, about 4×SSC to about 5×SSC, or about 5×SSC to about 6×SSC). In some embodiments, the SSC is present in the SSC hybridization buffer from about 2×SSC to about 4×SSC. In some embodiments, SSPE hybridization buffer can be used.

In some embodiments, the SSC hybridization buffer comprises a solvent. In some embodiments, the solvent comprises ethylene carbonate instead of formamide (2020, Kalinka et al., Scientia Agricola 78(4):c20190315). In some embodiments, ethylene carbonate is present in the SSC hybridization buffer from about 10% (w/v) to about 25% (w/v) (e.g., about 10% (w/v) to about 20% (w/v), about 10% (w/v) to about 15% (w/v), about 15% (w/v) to about 25% (w/v), about 15% (w/v) to about 20% (w/v), or about 20% (w/v) to about 25% (w/v)). In some embodiments, ethylene carbonate is present in the SSC hybridization buffer from about 15% (w/v) to about 20% (w/v). In some embodiments, ethylene carbonate is present in the SSC hybridization buffer at about 10% (w/v), about 11% (w/v), about 12% (w/v), about 13% (w/v), about 14% (w/v), about 15% (w/v), about 16% (w/v), about 17% (w/v), about 18% (w/v), about 19% (w/v), about 20% (w/v), about 21% (w/v), about 22% (w/v), about 23% (w/v), about 24% (w/v), or about 25% (w/v). In some embodiments, ethylene carbonate is present in the SSC hybridization buffer at about 13% (w/v).

In some embodiments, the SSC hybridization buffer is at a temperature from about 40° ° C. to about 60° ° C. e.g., about 40° ° C. to about 55° C., about 40° C. to about 50° C., about 40° C. to about 45° C., about 45° C. to about 60° C., about 45° ° C. to about 55° C., about 45° C. to about 50° C., about 50° ° C. to about 60° ° C., about 50° C. to about 55° C., or about 55° C. to about 60° C.). In some embodiments, the SSC hybridization buffer is at temperature from about 45° C. to about 55° C., or any of the subranges described herein. In some embodiments, the SSC hybridization buffer is at a temperature of about 40° C., about 41° C., about 42° C., about 43° C., about 44° C., about 45° C., about 46° C., about 47° C., about 48° C., about 49° ° C., about 50° C., about 51° C., about 52° ° C., about 53° ° C., about 54° C., about 55° C., about 56° C., about 57° C., about 58° C., about 59° C., or about 60° C. In some embodiments, the SSC hybridization buffer is at a temperature of about 50° C.

In some embodiments, the SSC hybridization buffer further comprises one or more of a carrier, a crowder, or an additive. Non-limiting examples of a carrier that can be included in the hybridization buffer include: yeast tRNA, salmon sperm DNA, lambda phage DNA, glycogen, and cholesterol. Non-limiting examples of a molecular crowder that can be included in the hybridization buffer include: Ficoll, dextran, Denhardt's solution, and PEG. Non-limiting examples of additives that can be included in the hybridization buffer include: binding blockers, RNase inhibitors, Tm adjustors and adjuvants for relaxing secondary nucleic acid structures (e.g., betaine, TMAC, and DMSO). Further, a hybridization buffer can include detergents such as SDS, Tween, Triton-X 100, and sarkosyl (e.g., N-Lauroylsarcosine sodium salt). A skilled artisan would understand that a buffer for hybridization of nucleic acids could include many different compounds that could enhance the hybridization reaction.

### (h) Washing

In some embodiments, the methods disclosed herein also include a wash step. The wash step removes any unbound probes. Wash steps could be performed between any of the steps in the methods disclosed herein. For example, a wash step can be performed after adding probes (e.g., any of the templated ligation probe pairs described herein) to the biological sample. As such, free/unbound probes are washed away, leaving only probes that have hybridized to an analyte. In some instances, multiple (i.e., at least 2, 3, 4, 5, or more) wash steps occur between the methods disclosed herein. Wash steps can be performed at times (e.g., 1, 2, 3, 4, or 5 minutes) and temperatures (e.g., room temperature; 4° C. known in the art and determined by a person of skill in the art.

In some instances, wash steps are performed using a wash buffer. In some instances, the wash buffer includes SSC (e.g., 1×SSC). In some instances, the wash buffer includes PBS (e.g., 1×PBS). In some instances, the wash buffer includes PBST (e.g., 1×PBST). In some instances, the wash buffer can also include formamide or be formamide frec.

Additional embodiments regarding wash steps are provided herein.

### (i) Formamide Free Wash Buffer

In some embodiments, after ligating a first probe and a second probe, the one or more unhybridized first probes, one or more unhybridized second probes, or both, are removed from the array. In some embodiments, after ligating a first probe, a second probe, and a third oligonucleotide, the one or more unhybridized first probes, one or more unhybridized second probes, or one or more third oligonucleotides, or all the above, are removed from the array.

In some embodiments, a pre-hybridization buffer is used to wash the sample. In some embodiments, a phosphate buffer is used. In some embodiments, multiple wash steps are performed to remove unbound oligonucleotides.

In some embodiments, removing includes washing the one or more unhybridized probes (e.g., a first probe, a second probe, and a third oligonucleotide) from the array in a formamide-free wash buffer.

In some embodiments, the formamide-free wash buffer is an SSC wash buffer. In some embodiments, SSC is present in the SSC wash buffer from about 0.01×SSC to about 1×SSC (e.g., about 0.01×SSC to about 0.5×SSC, 0.01×SSC to about 0.1×SSC, about 0.01×SSC to about 0.05×SSC, about 0.05×SSC to about 1×SSC, about 0.05×SSC to about 0.5×SSC, about 0.05×SSC to about 0.1×SSC, about 0.1×SSC to about 1×SSC, about 0.1×SSC to about 0.5×SSC, or about 0.5×SSC to about 1×SSC). In some embodiments, SSC is present in the SSC wash buffer at about 0.01×SSC, about 0.02×SSC, about 0.03×SSC, about 0.04×SSC, about 0.05×SSC, about 0.06×SSC, about 0.07×SSC, about 0.08×SSC, about 0.09×SSC, about 0.1×SSC, about 0.2×SSC, about 0.3×SSC, about 0.4×SSC, about 0.5×SSC, about 0.6×SSC, about 0.7×SSC, about 0.8×SSC, about 0.9×SSC, or about 0.1×SSC. In some embodiments, SSC is present in the SSC wash buffer at about 0.1×SSC.

In some embodiments, the SSC wash buffer comprises a detergent. In some embodiments, the detergent comprises sodium dodecyl sulfate (SDS). In some embodiments, SDS is present in the SSC wash buffer from about 0.01% (v/v) to about 0.5% (v/v) (e.g., about 0.01% (v/v) to about 0.4% (v/v), about 0.01% (v/v) to about 0.3% (v/v), about 0.01% (v/v) to about 0.2% (v/v), about 0.01% (v/v) to about 0.1% (v/v), about 0.05% (v/v) to about 0.5% (v/v), about 0.05% (v/v) to about 0.4% (v/v), about 0.05% (v/v) to about 0.3% (v/v), about 0.05% (v/v) to about 0.2% (v/v), about 0.05% (v/v) to about 0.1% (v/v), about 0.1% (v/v) to about 0.5% (v/v), about 0.1% (v/v) to about 0.4% (v/v), about 0.1% (v/v) to about 0.3% (v/v), about 0.1% (v/v) to about 0.2% (v/v), about 0.2% (v/v) to about 0.5% (v/v), about 0.2% (v/v) to about 0.4% (v/v), about 0.2% (v/v) to about 0.3% (v/v), about 0.3% (v/v) to about 0.5% (v/v), about 0.3% (v/v) to about 0.4% (v/v), or about 0.4% (v/v) to about 0.5% (v/v)). In some embodiments, the SDS is present the SSC wash buffer at about 0.01% (v/v), about 0.02% (v/v), about 0.03% (v/v), about 0.04% (v/v), about 0.05% (v/v), about 0.06% (v/v), about 0.07% (v/v), about 0.08% (v/v), about 0.09% (v/v), about 0.10% (v/v), about 0.2% (v/v), about 0.3% (v/v), about 0.4% (v/v), or about 0.5% (v/v), In some embodiments, the SDS is present in the SSC wash buffer at about 0.1% (v/v). In some embodiments, sarkosyl may be present in the SSC wash buffer.

In some embodiments, the SSC wash buffer comprises a solvent. In some embodiments, the solvent comprises formamide or ethylene carbonate. In some embodiments, ethylene carbonate is present in the SSC wash buffer from about 10% (w/v) to about 25% (w/v), or any of the subranges described herein. In some embodiments, ethylene carbonate is present in the SSC wash buffer from about 15% (w/v) to about 20% (w/v). In some embodiments, ethylene carbonate is present in the SSC wash buffer at about 16% (w/v).

In some embodiments, the SSC wash buffer is at a temperature from about 50° C. to about 70° ° C. e.g., about 50° C. to about 65° C., about 50° C. to about 60° C., about 50° C. to about 55° C., about 55° ° C. to about 70° ° C., about 55° C. to about 65° C., about 55° ° C. to about 60° C., about 60° ° C. to about 70° C., about 60° C. to about 65° C., or about 65° C. to about 70° ° C. In some embodiments, the SSC wash buffer is at a temperature from about 55° C. to about 65° C. In some embodiments, the SSC wash buffer is at a temperature about 50° C., about 51° C., about 52° C., about 53° ° C., about 54° C., about 55° C., about 56° C., about 57° C., about 58° C., about 59° C., about 60° ° C., about 61° ° C., about 62° ° C., about 63° ° C., about 64° C., about 65° C., about 66° C., about 67° C., about 68° C., about 69° C., or about 70° ° C. In some embodiments, the SSC wash buffer is at a temperature of about 60° ° C.

In some embodiments, the method includes releasing the ligation product, where releasing is performed after the array is washed to remove the one or more unhybridized first and second probes.

### (i) Ligation

In some embodiments, after hybridization of the probe oligonucleotides (e.g., a first probe, a second probe, and/or a third oligonucleotide) to the analyte comprising a genetic variant, the probes (e.g., a first probe, a second probe, and/or a third oligonucleotide) can be ligated together, creating a single ligation product that includes one or more sequences that are complementary to the analyte. Ligation can be performed enzymatically or chemically, as described herein.

In some instances, the ligation is an enzymatic ligation reaction, using a ligase (e.g., T4 RNA ligase (Rnl2), a SplintR ligase, a single stranded DNA ligase, or a T4 DNA ligase). Scc, e.g., Zhang et al.; RNA Biol. 2017; 14(1): 36-44, which is incorporated by reference in its entirety, for a description of KOD ligase. Following the enzymatic ligation reaction, the probes (e.g., a first probe, a second probe, and/or a third oligonucleotide) may be considered ligated.

In some embodiments, a polymerase catalyzes synthesis of a complementary strand of the ligation product, creating a double-stranded ligation product. In some instances, the polymerase is DNA polymerase. In some embodiments, the polymerase has 5′ to 3′ polymerase activity. In some embodiments, the polymerase has 3′ to 5′ exonuclease activity for proofreading. In some embodiments, the polymerase has 5′ to 3′ polymerase activity and 3′ to 5′ exonuclease activity for proofreading.

In some embodiments, the probe (e.g., a first probe, a second probe, and/or a third oligonucleotide) may each comprise a reactive moiety such that, upon hybridization to the target and exposure to appropriate ligation conditions, the probe oligonucleotides may ligate to one another. In some embodiments, probe oligonucleotides that include a reactive moiety are ligated chemically. For example, a first probe capable of hybridizing to a first target region (e.g., a first target sequence or a first portion) of a nucleic acid molecule may comprise a first reactive moiety, and a second probe oligonucleotide capable of hybridizing to a second target region (e.g., a second target sequence or a second portion) of the nucleic acid molecule may comprise a second reactive moiety. When the first and second probes are hybridized to the first and second target regions (e.g., first and second target sequences) of the nucleic acid molecule, the first and second reactive moieties may be adjacent to one another. A reactive moiety of a probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phorphorothioate), acids, amines, and phosphates. For example, the first reactive moiety of a first probe may comprise an azide moiety, and a second reactive moiety of a second probe may comprise an alkyne moiety. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a thiol-ene reaction; a nucleophilic substation reaction; or another reaction. In some cases, reaction between the first and second reactive moieties may yield a triazole moiety or an isoxazoline moiety. A reaction between the first and second reactive moieties may involve subjecting the reactive moicties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first probe hybridized to a first target region (e.g., a first target sequence or first portion) of the nucleic acid molecule and a second reactive moiety of a third probe oligonucleotide hybridized to a second target region (e.g., a first target sequence or a first portion) of the nucleic acid molecule may link the first probe and the second probe to provide a ligated probe. Upon linking, the first and second probe may be considered ligated. Accordingly, reaction of the first and second reactive moicties may comprise a chemical ligation reaction such as a copper-catalyzed 5′ azide to 3′ alkyne “click” chemistry reaction to form a triazole linkage between two probe oligonucleotides. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond.

In some instances, ligation is performed in a ligation buffer. In some instances, one probe comprises a diribo-moiety on its 3′ end and the other probe comprises a phosphorylated group on the 5′ end. In instances where probe ligation is performed on diribo-containing probes, the ligation buffer can include T4 RNA Ligase Buffer 2, enzyme (e.g., RNL2 ligasc), and nuclease free water. In instances where probe ligation is performed on DNA probes, the ligation buffer can include Tris-HCl pH7.5, MnCl₂, ATP, DTT, surrogate fluid (e.g., glycerol), enzyme (e.g., SplintR ligase), and nuclease-free water.

In some embodiments, the ligation buffer includes additional reagents. In some instances, the ligation buffer includes adenosine triphosphate (ATP) is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers the AMP to the phosphorylated 5′-end at the nick, forming a 5′-5′ pyrophosphate bond. Finally, the ligase catalyzes an attack on this pyrophosphate bond by the OH group at the 3′-end of the nick, thereby sealing it, whereafter ligase and AMP are released. If the ligase detaches from the substrate before the 3′ attack, e.g., because of premature AMP reloading of the enzyme, then the 5′ AMP is left at the 5′-end, blocking further ligation attempts. In some instances, ATP is added at a concentration of about 1 μM, about 10 μM, about 100 μM, about 1000 μM, or about 10000 μM during the ligation reaction.

In some embodiments, cofactors that aid in joining of the probe oligonucleotides are added during the ligation process. In some instances, the cofactors include magnesium ions (Mg²⁺). In some instances, the cofactors include manganese ions (Mn²⁺). In some instances, Mg²⁺ is added in the form of MgCl₂. In some instances, Mn²⁺ is added in the form of MnCl₂. In some instances, the concentration of MgCl₂ is at about 1 mM, at about 10 mM, at about 100 mM, or at about 1000 mM. In some instances, the concentration of MnCl₂ is at about 1 mM, at about 10 mM, at about 100 mM, or at about 1000 mM.

In some embodiments, the ligation product includes a capture probe capture domain, which can bind to a capture probe (e.g., a capture probe immobilized, directly or indirectly, on a substrate). In some embodiments, methods provided herein include contacting a biological sample with a substrate, wherein the capture probe is affixed to the substrate (e.g., immobilized to the substrate, directly or indirectly). In some embodiments, the capture probe capture domain of the ligated probe specifically binds to the capture domain.

After ligation, in some instances, the biological sample is washed with a post-ligation wash buffer. In some instances, the post-ligation wash buffer includes one or more of SSC (e.g., 1×SSC), ethylene carbonate or formamide, and nuclease free water. In some instances, the biological sample is washed at this stage at about 50° C. to about 70° ° C. In some instances, the biological sample is washed at about 60° C.

### (i) Ligation Including Pre-Adenylated 5′ Phosphate on Second Probe

Provided herein are methods for determining the presence and location of a variant in a target nucleic acid in a biological sample that include: (a) contacting the biological sample with a substrate comprising a plurality of capture probes, where a capture probe of the plurality of capture probes comprises a capture domain and a spatial barcode; (b) hybridizing a target nucleic acid in the biological sample with a first probe and a second probe, where the first probe comprises, from 3′ to 5′, a sequence substantially complementary to the capture domain and a sequence that is substantially complementary to a first sequence in the target nucleic acid and has a pre-adenylated phosphate group at its 5′ end; the second probe comprises a sequence substantially complementary to a second sequence in the target nucleic acid. It is appreciated that one or both probes can be designed to hybridize to a genetic variant of interest. In some instances, after step (b) is performed, the method further includes (c) generating a ligation product by ligating a 3′ end of the second probe to the 5′ end of the first probe using a ligase that does not require adenosine triphosphate for ligase activity; (d) releasing the ligation product from the target nucleic acid and binding the capture domain of the ligation product specifically to the capture domain of capture probe; and (c) determining (i) all or a part of a sequence corresponding to the ligation product, or a complement thereof, and (ii) all or a part of a sequence corresponding to the spatial barcode, or a complement thereof, and using the determined sequences of (i) and (ii) to identify the location of the target nucleic acid in the biological sample

In some instances, the ligase that does not require adenosine triphosphate for ligasc activity (e.g., thermostable 5′ AppDNA/RNA Ligase, truncated T4 RNA Ligase 2 (trRnl2), truncated T4 RNA Ligase 2 K227Q, truncated T4 RNA Ligase 2 KQ, Chlorella Virus PBCV-1 DNA Ligase, and combinations thereof). Sec, e.g., Nichols et al., “RNA Ligases,” Curr. Protocol. Molec. Biol. 84(1):3.15.1-. 4 (2008); Viollet et al., “T4 RNA Ligase 2 Truncated Active Site Mutants: Improved Tools for RNA Analysis,” BMC Biotechnol. 11: 72 (2011); and Ho et al., “Bacteriophage T4 RNA Ligase 2 (gp24.1) Exemplifies a Family of RNA Ligases Found in All Phylogenetic Domains,” PNAS 99(20): 12709-14 (2002), which are hereby incorporated by reference in their entirety for a description of T4 RNA Ligases and truncated T4 RNA Ligases. Thermostable 5′ AppDNA/RNA Ligase is an enzyme belonging to the Ligase family that catalyzes the ligation of the 3′ end of ssRNA or ssDNA to a 5′-adenylated ssDNA or 5′-adenylated ssRNA. Truncated T4 RNA Ligase 2 is an enzyme belonging to the Ligase family that catalyzes the ligation of dsRNA nicks and ssRNA to ssRNA. It can also ligate the 3′ end of RNA or DNA to a 5′-pDNA when annealed to an RNA complement, and the 3′ end of RNA to a 5′-pRNA when annealed to a DNA complement, with reduced efficiency. Truncated T4 RNA Ligase 2 K227Q is an enzyme belonging to the Ligase family that catalyzes the ligation of the 3′ end of ssRNA to 5′ adenylated ssDNA and 5′ adenylated ssRNA. It has a reduction of side products as compared to truncated T4 RNA Ligase 2. Truncated T4 RNA Ligase 2 KQ is an enzyme belonging to the Ligase family that catalyzes the ligation of the 3′ end of ssRNA to 5′ adenylated ssDNA and 5′ adenylated ssRNA. It is a preferred choice for ligation of ssRNA to preadenylated adapters and has a reduction of side products as compared to truncated T4 RNA Ligase 2.

In some embodiments, the T4 RNA Ligase comprises a K227Q mutation. See Viollet et al., “T4 RNA Ligase 2 Truncated Active Site Mutants: Improved Tools for RNA Analysis,” BMC Biotechnol. 11, which is hereby incorporated by reference in its entirety.

In some instances, cofactors that aid in ligation of the first and second probe are added during ligation. In some instances, the cofactors include magnesium ions (Mg²⁺). In some instances, the cofactors include manganese ions (Mn²⁺). In some instances, Mg²⁺ is added in the form of MgCl₂. In some instances, Mn²⁺ is added in the form of MnCl₂. In some instances, the concentration of MgCl₂ is at about 1 mM to about 10 mM. In some instances, the concentration of MnCl₂ is at about 1 mM to about 10 mM.

In some instances, the ligation occurs at a pH in the range of about 6.5 to about 9.0, about 6.5 to about 8.0, or about 7.5 to about 8.0.

In some embodiments, the ligation buffer includes an enzyme storage buffer. In some embodiments, the enzymes storage buffer includes glycerol. In some embodiments, the ligation buffer is supplemented with glycerol. In some embodiments, the glycerol is present in the ligation buffer at a total volume of 15% v/v.

### (j) Permeabilization and Releasing the Ligation Product

In some embodiments, the methods provided herein include a permeabilizing step in order to release the ligated probe that now comprises a sequence that includes the complement of a genetic variant. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, after creating a ligation product (e.g., by ligating a first probe and a second probe that are hybridized to adjacent sequences in the analyte), the biological sample is permeabilized. In some embodiments, the biological sample is permeabilized contemporaneously with or prior to contacting the biological sample with a first probe and a second probe, hybridizing the first probe and the second probe to the analyte, generating a ligation product by ligating the first probe and the second probe, and releasing the ligated product from the analyte.

In some embodiments, methods provided herein include permeabilization of the biological sample such that the capture probe can more easily bind to the captured ligated probe (i.e., compared to no permcabilization).

In some instances, the permeabilization step includes application of a permeabilization buffer to the biological sample. In some instances, the permeabilization buffer includes a buffer (e.g., Tris pH 7.5), MgCl2, sarkosyl detergent (e.g., sodium lauroyl sarcosinate), enzyme (e.g., proteinase K), and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

In some embodiments, after generating a ligation product, the ligation product is released from the analyte. In some embodiments, a ligation product is released from the analyte using an endoribonuclease. In some embodiments, the endoribonuclease is RNase H, RNase A, RNase C, or RNase I. In some embodiments, the endoribonuclease is RNase H. RNase H is an endoribonuclease that specifically hydrolyzes the phosphodiester bonds of RNA, when hybridized to DNA. RNase H is part of a conserved family of ribonucleases which are present in many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA:DNA heteroduplex. In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli.

In some instances, the releasing step is performed using a releasing buffer. In some instances, the release buffer includes one or more of a buffer (e.g., Tris pH 7.5), enzyme (e.g., RNAse H) and nuclease-free water. In some instances, the releasing step is performed at 37° C. In some instances, the releasing step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 30 minutes.

In some instances, the releasing step occurs before the permeabilization step. In some instances, the releasing step occurs after the permeabilization step. In some instances, the releasing step occurs at the same time as the permeabilization step (e.g., in the same buffer).

### (k) Biological Samples

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells. In some instances, the biological sample comprises nucleic acids with one or more genetic variants. In some instances, the one or more genetic variants is associated with a disease or disease state.

In some instances, the cancer is breast cancer. In some instances, the breast cancer is triple positive breast cancer (TPBC). In some instances, the breast cancer is triple negative breast cancer (TNBC).

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not cosin, when location of a cell nucleus is needed.

In some embodiments, a biological sample (e.g., tissue section) can be fixed with methanol, stained with hematoxylin and cosin, and imaged. In some embodiments, fixing, staining, and imaging occurs before one or more probes are hybridized to the sample. Some embodiments of any of the workflows described herein can further include a destaining step (e.g., a hematoxylin and cosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA.

In some embodiments, permeabilizing a sample includes washing the sample with a phosphate buffer. In some embodiments, the buffer is PBS. In some embodiments, the buffer is PBST.

(l) Determining the Sequence of the Ligation Product and/or the 5′ Flap

After a target analyte proxy such as a ligation product comprising a 5′ Flap has hybridized or otherwise been associated with a capture probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein) in order to create a copy of the ligated probe. This process involves synthesis of a complementary strand of the ligated probe, e.g., generating a full length complement of the ligation product. In some embodiments, the capture probe is extended after hybridizing a 5′ Flap sequence to the capture domain. In some embodiments, the capture probe is extended after hybridization of the ligation product that results from the cleavage of the 5′ Flap sequence from the tripartite structure and ligation of the analyte specific probes (e.g., first and second oligonucleotide probes as shown in FIG. 7A). In some embodiments, the capture probe is extended using one or more DNA polymerases.

In some embodiments, a capture domain of a capture probe includes a primer for producing the complementary strand of a ligated probe hybridized to the capture probe, e.g., a primer for DNA polymerase. The nucleic acid, e.g., DNA and/or ligated probes or complement thereof, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA molecule is generated. In some embodiments, a “full-length” DNA molecule refers to the whole of the captured ligated product. For example, the full length DNA molecule refers to the whole of the ligation product released following RNase H digestion of the RNA from the RNA:DNA duplex.

In some embodiments, double-stranded extended capture probes (now containing the ligated probe) are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes containing the complement of the ligated probe are amplified to yield quantities that are sufficient for analysis, e.g., via DNA sequencing. In some embodiments, the extended ligation products (e.g., the capture probe plus the ligated probe) act as a template for the amplification reaction (e.g., a polymerase chain reaction).

In some embodiments, the amplification reaction incorporates an affinity group onto the extended ligation product using a primer including the affinity group. In some embodiments, the primer includes an affinity group and the extended ligation product includes the affinity group. The affinity group can correspond to any of the affinity groups described previously.

In some embodiments, the extended ligation products include the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended ligation products can function to release the products from the substrate, insofar as copies of the extended ligation products are not immobilized on the substrate.

In some embodiments, the extended ligation product or complement or amplicon thereof is released. The step of releasing from the surface of the substrate can be achieved in a number of ways. In some embodiments, an extended ligation product or a complement thereof is released from the array by nucleic acid cleavage and/or by denaturation (e.g., by heating to denature a double-stranded molecule).

In some embodiments, the extended ligation product or complement or amplicon thereof is released from the surface of the substrate (e.g., array) by physical means. For example, where the extended ligation product is indirectly immobilized on the array substrate, e.g., via hybridization to a surface probe, it can be sufficient to disrupt the interaction between the extended ligation product and the surface probe. Methods for disrupting the interaction between nucleic acid molecules include denaturing double stranded nucleic acid molecules are known in the art. A straightforward method for releasing the DNA molecules (i.e., of stripping the array of extended products) is to use a solution that interferes with the hydrogen bonds of the double stranded molecules. In some embodiments, the extended capture product is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended ligation product from the substrate.

In some embodiments, probes complementary to the extended ligation product can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended ligation product can be washed away. In some embodiments, probes complementary to the extended ligation product can be detected on the substrate (e.g., imaging, any of the detection methods described herein).

In some embodiments, probes complementary to an extended ligation products can be about 4 nucleotides to about 100 nucleotides long. In some embodiments, probes (e.g., detectable probes) can be about 10 nucleotides to about 90 nucleotides long. In some embodiments, probes (e.g., detectable probes) can be about 20 nucleotides to about 80 nucleotides long. In some embodiments, probes (e.g., detectable probes) can be about 30 nucleotides to about 60 nucleotides long. In some embodiments, probes (e.g., detectable probes) can be about 40 nucleotides to about 50 nucleotides long. In some embodiments, probes (e.g., detectable probes) can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended ligation product. In some embodiments, about 1 to about 10 probes can be contacted to the substrate and specifically bind (e.g., hybridize) to an extended ligation product. In some embodiments, about 10 to about 100 probes can be contacted to the substrate and specifically bind (e.g., hybridize) to an extended ligation product. In some embodiments, about 20 to about 90 probes can be contacted to the substrate. In some embodiments, about 30 to about 80 probes (e.g., detectable probes) can be contacted to the substrate. In some embodiments, about 40 to about 70 probes can be contacted to the substrate. In some embodiments, about 50 to about 60 probes can be contacted to the substrate. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended ligation product.

In some embodiments, the probes can be complementary to a ligation product of a single analyte (e.g., a single gene). In some embodiments, the probes can be complementary to one or more analyte ligation products (e.g., analytes in a family of genes). In some embodiments, the probes (e.g., detectable probes) can be directed to ligation products for a panel of genes associated with a disease (e.g., cancer, Alzheimer's disease, Parkinson's disease).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### (m) Kits

In some embodiments, also provided herein are kits that include one or more reagents to detect one or more analytes described herein. In some instances, the kit includes a substrate comprising a plurality of capture probes comprising a spatial barcode and the capture domain. In some instances, the kit includes a plurality of probes (e.g., a first probe oligonucleotide and a second probe oligonucleotide).

A non-limiting example of a kit used to perform any of the methods described herein includes: (a) a substrate comprising a plurality of capture probes comprising a spatial barcode and a capture domain; (b) a system comprising: a first probe oligonucleotide and a second probe oligonucleotide, wherein the first probe oligonucleotide and the second probe oligonucleotide each comprise a sequence that is substantially complementary to adjacent sequences of the analyte, wherein the first probe oligonucleotide and the second probe oligonucleotide are capable of forming an invasive cleavage structure in the presence of the analyte, wherein the second probe oligonucleotide comprises a capture probe binding domain; and (c) instructions for performing the method of any one of the preceding claims.

A non-limiting example of a kit used to perform any of the methods described herein includes: (a) a substrate comprising a plurality of capture probes comprising a spatial barcode and a capture domain; (b) a system comprising: a first probe oligonucleotide and a second probe oligonucleotide, wherein the first probe oligonucleotide and the second probe oligonucleotide each comprise a sequence that is substantially complementary to sequences of the analyte where there is a gap between the sequences of the analyte, wherein the first probe oligonucleotide and the second probe oligonucleotide are capable of forming an invasive cleavage structure in the presence of the analyte, wherein the second probe oligonucleotide comprises a capture probe binding domain; and (c) instructions for performing the method of any one of the preceding claims.

In some embodiments of any of the kits, the first probe oligonucleotide further includes at least one nucleotide that is complementary to a wild-type sequence of the genetic variant.

In some embodiments of any of the kits, the second probe oligonucleotide further comprises: (i) a sequence of non-complementary nucleotides; (ii) a nucleotide complementary to the genetic variant; and (iii) a capture probe binding domain.

In some embodiments of any of the kits, the kit includes an endonuclease. In some embodiments, the endonuclease cleaves the invasive cleavage structure. In some embodiments, the endonuclease is Flap endonuclease 1 (FEN1).

In some embodiments of any of the kits, the kit includes a reverse transcriptase. In some embodiments, the reverse transcriptase includes a Moloney Murine Leukemia Virus (M-MulV).

In some embodiments of any of the kits described herein, the kit includes a second probe that includes a preadenylated phosphate group at its 5′ end and a first probe comprising at least two ribonucleic acid bases at the 3′ end.

### EXAMPLES

### Example 1—Single Nucleotide Polymorphism Detection

This example provides an exemplary method for determining the presence or absence of a genetic variant at a spatial location in a biological sample. In a non-limiting example, a first probe oligonucleotide having a non-complementary nucleotide overlapping the target sequence (e.g., genetic variant) and a second probe oligonucleotide having a sequence complementary to the target sequence (e.g., genetic variant) are used to determine the presence or absence of a genetic variant at a spatial location in a biological sample. Others have demonstrated SNP detection using invader assays. Non-limiting aspects of SNP detection with invader assays are described in U.S. Pat. Nos. 7,011,944, 6,913,881, 6,875,572 and 6,872,816, each of which is incorporated by reference in its entirety and each of which can be used herein in any combination.

As shown in FIG. 7A, an mRNA molecule 701 that includes a single nucleotide polymorphism (SNP) (i.e., genetic variant) at a target sequence is contacted with a first probe oligonucleotide 703 and a second probe oligonucleotide 704. The first probe oligonucleotide 703 includes a function sequence 705 (e.g., R2 sequence), a sequence 706 that is substantially complementary to a sequence that is 3′ of a target sequence (e.g., genetic variant) and a non-complementary nucleotide 707 that is overlapping the target sequence (e.g., genetic variant). The second probe oligonucleotide includes a 5′ Flap sequence 708 (e.g., non-complementary nucleotide sequence), a nucleotide 709 that is complementary to the target sequence (e.g., genetic variant), a sequence 710 that is substantially complementary to a sequence 5′ of the target sequence (e.g., genetic variant), and a capture probe binding domain 711 that is capable of binding to a capture domain of a capture probe. The first probe oligonucleotide and the second probe oligonucleotide hybridize 712 to the mRNA molecule. Excess first and second probes are preferably washed off.

In the presence of the genetic variant (e.g., SNP) and where the first probe oligonucleotide includes a non-complementary sequence 707 that overlaps with the target sequence and the second probe oligonucleotide includes a sequence 709 that overlaps with the target sequence, the first probe oligonucleotide and the second probe oligonucleotide are capable of forming an invasive cleavage structure 713, which can be cleaved by a FEN1 endonuclease 714. When the sequence of the second probe oligonucleotide is not complementary to the target sequence (e.g., genetic variant) or the first probe oligonucleotide includes a complementary sequence that overlaps the target sequence (e.g., genetic variant), no invasive cleavage structure is formed and FEN1-mediated cleave will not occur.

A FEN1 endonuclease then cleaves 715 the 5′ Flap if the appropriate conditions described above are met, thereby removing the non-complementary nucleotides (e.g., the 5′ Flap) from the second probe oligonucleotide. Following FEN1-mediated cleavage, the first probe oligonucleotide and the second probe oligonucleotide are ligated 716, thereby creating a ligated probe 717 that is substantially complementary to the mRNA molecule (e.g., a proxy that is complementary to the target mRNA and the SNP).

The biological sample is contacted with a substrate that includes capture probes affixed to the substrate, where the capture probes include a spatial barcode and the capture domain. The ligated probe that is bound to the mRNA molecule is released from the mRNA molecule using RNaseH. Following release of the ligated probe, the capture probe binding domain of the ligated probe specifically binds to the capture domain on the capture probe, thereby capturing the ligated probe on the substrate. Finally, all or part of the sequence of the ligated probe specifically bound to the capture domain along with the all or part of the sequence of the spatial barcode of the capture probe is sequenced and used to determine the presence or absence of a single nucleotide polymorphism (SNP) at the target sequence.

### Example 2—Identifying a Location of an Analyte in a Biological Sample Using Templated Ligation and Gap Filling

This example provides an exemplary method for determining the location of an analyte in a biological sample using templated ligation and gap filling. In a non-limiting example, a first probe oligonucleotide having a sequence that is complementary to a sequence 3′ of the target sequence and a second probe oligonucleotide having a sequence that is complementary to a sequence 5′ of the target sequence are used to determine the location of an analyte in a biological sample.

As shown in FIG. 7B, an mRNA molecule 701 is contacted with a first probe oligonucleotide 703 and a second probe oligonucleotide 704. The first probe oligonucleotide 703 that includes a functional sequence 705 (e.g., a R2 sequence) and a sequence 706 that is substantially complementary to a sequence that is 3′ of a target sequence. The second probe oligonucleotide 704 includes a 5′ Flap sequence 708 (e.g., non-complementary nucleotide sequence), a sequence 710 that is substantially complementary to a sequence 5′ of the target sequence, and a capture probe binding domain 711 that is a capable of binding to a capture domain of a capture probe. In this example, the sequences in the mRNA molecule to which the first probe and the second probe were complementary are not directly adjacent to each other on the mRNA molecule (e.g., there is a “gap” 720 of unbound nucleic acids between the first probe oligonucleotide and the second probe oligonucleotide). The first probe oligonucleotide 703 and the second probe oligonucleotide 704 hybridize 712 to the mRNA molecule. Excess first and second probes are preferably washed off. The first probe oligonucleotide is extended 721 using an M-MulV reverse transcriptase enzyme in order to generate an extended first probe oligonucleotide. The first probe oligonucleotide is extended 722 up to the sequence that is adjacent to the where the second probe oligonucleotide is bound to the RNA sequence.

A FEN1 endonuclease is added 723 to cleave the 5′ Flap. The FEN1 endonuclease 724 cleaves the 5′ Flap removing the non-complementary nucleotides from the second probe oligonucleotide including nucleotides 725 of the second probe oligonucleotide that were displaced by the M-MulV when generating the extended first probe oligonucleotide. Following FEN1-mediated cleavage, the extended first probe oligonucleotide and the second probe oligonucleotide are ligated 726, thereby creating a ligated probe 727 that is substantially complementary to the mRNA molecule.

The biological sample is then processed according to the same steps as described in Example 1. Briefly, the biological sample is contacted with a substrate, the ligated probe that is bound to the mRNA molecule is released, the capture probe binding domain of the ligated probe specifically binds to the capture domain on the capture probe, and all or part of the sequence of the ligated probe and the spatial barcode are sequenced in order to determine the location of the analyte in the biological sample.

### Example 3—Identifying a Location of an Analyte in a Biological Sample Using Templated Ligation and Gap Probes

This example provides an exemplary method for determining the location of an analyte having one or more genetic variants in a biological sample using templated ligation and gap probes. In a non-limiting example, a first probe oligonucleotide having a sequence that is complementary to a sequence 3′ of the target sequence, a second probe oligonucleotide having a sequence that is complementary to a sequence 5′ of the target sequence, and a gap probe oligonucleotide are used to determine the location of an analyte in a biological sample.

As seen in FIG. 7C, an mRNA molecule 701 having either a wild type nucleotide (left panel) or a genetic variant (right panel) located at a target sequence is contacted with a first probe oligonucleotide 703 and a second probe oligonucleotide 704. The first probe oligonucleotide includes a functional sequence 705 (e.g., R2 sequence) and a sequence 706 that is substantially complementary to a sequence that is 3′ of a target sequence. The second probe oligonucleotide includes a sequence 710 that is substantially complementary to a sequence 5′ of the target sequence, and a capture probe binding domain 711 (e.g., a poly(A) sequence) that is a capable of binding to a capture domain of a capture probe (e.g., a poly(T) sequence). The complementary sequences of the first probe oligonucleotide and the second probe oligonucleotide are six nucleotides 730 away from each other on the mRNA molecule (e.g., a six nucleotide gap). This gap of six nucleotides includes the target sequence that includes either a wild type nucleotide 731 or includes a genetic variation 732 as compared to a wild type sequence.

The first probe oligonucleotide and the second probe oligonucleotide hybridize to the mRNA molecule. Excess first and second probes are preferably washed off. The biological sample is then contacted with a plurality of gap probe oligonucleotides 733. The plurality of gap probe oligonucleotides includes a pool of gap probe oligonucleotides where each gap probe oligonucleotide includes a different nucleic acid at the target sequence. Only a gap probe oligonucleotide that is complementary to the six nucleotide gap binds to the mRNA molecule between the first probe oligonucleotide and the second probe oligonucleotide (see, e.g., numerals 734 and 735 in FIG. 7C). Following binding of the gap probe oligonucleotide, a T4 RNA ligase 2 (Rn12) 736 ligates the first probe oligonucleotide to the 5′ end of the gap probe oligonucleotide and the second probe oligonucleotide to the 3′ end gap probe oligonucleotide.

The biological sample is processed according to the same steps as described in Example 1. Briefly, the biological is contacted with a substrate, the ligated probe that is bound to the mRNA molecule is released, the capture probe binding domain of the ligated probe specifically binds to the capture domain on the capture probe, and all or part of the sequence of the ligated probe and the spatial barcode are sequenced in order to determine the location of an mRNA molecule having one or more genetic variants in a biological sample.

### Example 4—Assessing 5′ Flap Methodologies for Variant Detection on a Spatial Array

In this example, a set of experiments was performed to assess a method for determining the presence or absence of a genetic variant at a spatial location in a biological sample using templated ligation and an invader strategy. In this example, a first probe oligonucleotide having a non-complementary nucleotide overlapping the target sequence (e.g., genetic variant) and a second probe oligonucleotide having a sequence complementary to the target sequence (e.g., genetic variant) were used to determine the presence or absence of a genetic variant at a spatial location in a biological sample: ssDNA (left panel) or RNA (right panel).

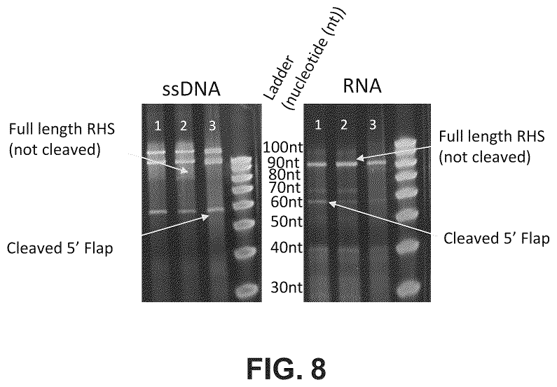

FIG. 8 shows a polyacrylamide gel (PAGE gel) of reaction products following a set of experiments as outlined, for example, in FIG. 7A. Briefly, the LHS (i.e., the first probe oligonucleotide) and RHS probes (i.e., the second probe oligonucleotide that includes the 5′ Flap) were incubated with either an ssDNA template (left panel) or RNA template (right panel), followed by cleavage of the 5′ Flap sequence with an endonuclease or a polymerase with 5′ endonuclease activity. Following hybridization, the 5′ Flap cleavage was mediated by cither (1) Tth Polymerase, (2) Taq Polymerase, or (3) FEN1. As shown in FIG. 8, the 5′ Flap was cleavable by all three of the enzymes used, wherein the cleavage efficiency appeared greater using an ssDNA template and to a lesser degree when using the RNA as a template.

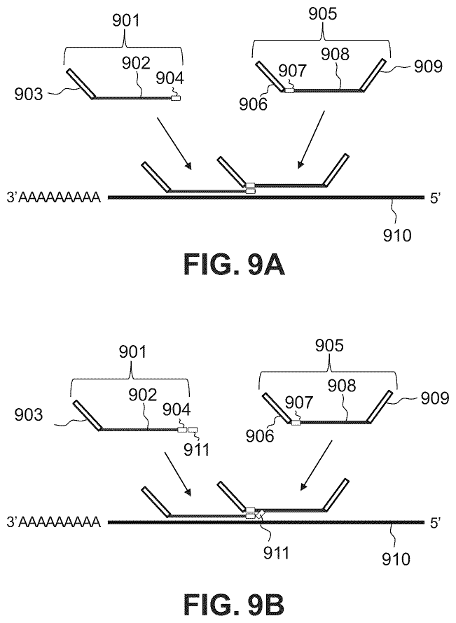

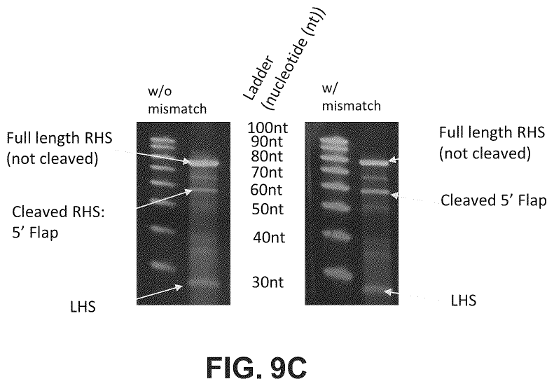

FIGS. 9A-9B show schematics comparing two experimental approaches for templated ligation combined with an invader assay. FIG. 9A shows an invasive cleavage structure (sec, e.g., numeral 713 in FIG. 7A) where a first probe oligonucleotide 901 includes a non-complementary sequence 902 that overlaps with the target sequence, a functional sequence 903, and a non-complementary nucleotide 904 that is overlapping the target sequence; a second probe oligonucleotide 905 includes a 5′ Flap sequence 906 (e.g., non-complementary nucleotide sequence), a nucleotide 907 that is complementary to the target sequence (e.g., genetic variant), a sequence 908 that is substantially complementary to a sequence 5′ of the target sequence (e.g., genetic variant), and a capture probe binding domain 909 that is capable of binding to a capture domain of a capture probe bind to the target nucleic acid 910. FIG. 9B shows an invasive cleavage structure where the first probe oligonucleotide includes a mismatched nucleotide 911 in addition to the elements in FIG. 9A and the second probe oligonucleotide that includes the same elements as shows in FIG. 9A. FIG. 9C shows a PAGE gel of reaction products comparing LHS probes (i.e., the first probe oligonucleotide) that include a mismatch on the 3′ end (FIG. 9C, right panel) or does not include a mismatch on the 3′ end (FIG. 9C, left panel). Surprisingly, 5′ Flap cleavage was more efficient when the 3′ end of the LHS included a mismatch (FIG. 9C, right panel) then without the mismatch (FIG. 9C, left panel).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the presence or absence of a genetic variant in an analyte at a spatial location in a biological sample comprising:
(a) contacting the biological sample with a first probe oligonucleotide and a second probe oligonucleotide, wherein the first probe oligonucleotide and the second probe oligonucleotide each comprise a sequence that is substantially complementary to adjacent sequences of the analyte, wherein the first probe oligonucleotide and the second probe oligonucleotide are capable of forming an invasive cleavage structure in the presence of the analyte, wherein the second probe oligonucleotide comprises a capture probe binding domain;
(b) hybridizing the first probe oligonucleotide and the second probe oligonucleotide to the analyte;
(c) ligating the first probe oligonucleotide and the second probe oligonucleotide, thereby creating a ligated probe;
(d) releasing the ligated probe from the analyte;
(e) contacting the ligated probe with a substrate comprising a plurality of capture probes, wherein a capture probe of the plurality comprises a spatial barcode and the capture domain;
(f) hybridizing the ligated probe to the capture domain; and
(g) determining (i) all or a part of the sequence of the ligated probe specifically bound to the capture domain, or a complement thereof, and (ii) all or a part of the sequence of the spatial barcode, or a complement thereof, and using the determined sequence of (i) and (ii) to identify the spatial location of the analyte in the biological sample.
